(19) United States
(12) Patent Application Publication
(10) Pub. No.: US 2021/0230607 A1
(43) Pub. Date: Jul. 29, 2021

(54) METHOD FOR RECONSTRUCTING COMPLEX BIOLOGICAL SYSTEM ON THE BASIS OF POLYPROTEIN, AND USE THEREOF IN HIGH ACTIVITY SUPER SIMPLIFIED NITROGEN FIXATION SYSTEM CONSTRUCTION

(71) Applicant: Peking University, Beijing (CN)

(72) Inventors: Jianguo Yang, Beijing (CN); Xiaqing Xie, Beijing (CN); Nan Xiang, Beijing (CN); Zhexian Tian, Beijing (CN); Ray Dixon, Norwich (GB); Yiping Wang, Beijing (CN)

(73) Assignee: Peking University, Beijing (CN)

(21) Appl. No.: 17/054,455

(22) PCT Filed: May 11, 2018
(86) PCT No.: PCT/CN2018/086490

§ 371 Date: Nov. 10, 2020

(51) Int. Cl. C12N 15/64; C12N 15/62

(52) U.S. Cl. CPC C12N 15/64; C12N 15/62

(57) ABSTRACT

An expression method, a vector and a vector composition are provided. In particular, a method for exogenously expressing a complex biological system in host cells, as well as a vector and a vector composition for the method are provided.

### TECHNICAL FIELD

The invention belongs to the field of bioengineering, and relates to a method of expressing a foreign gene in a host cell. In particular, the invention relates to a method for expressing a complex biological system (CBS) in a host cell, as well as vectors and vector compositions for expressing the CBS.

### BACKGROUND ART

A complex biological system (CBS) is a system constituted of multiple genes in an organism that encodes multiple components associated with specific functions or traits, such as nanomachines in an organism, obtaining nutrients and energy from various sources by an organism, metabolic pathways and biosynthesis of natural products, and the like. Genetic engineering of such systems with a large number of genetic components is often difficult, particularly as there is a stoichiometric requirement for balanced expression of the encoded protein components to achieve functions or traits associated with the system. To date, one approach towards engineering CBS involves the complete refactoring of each individual gene, in which all the original native regulatory components have been removed and artificially synthetic regulatory components have been added. The disadvantage of this approach is the increased fragility of refactored systems compared to native systems, and the relative expression levels of multiple proteins encoded by the refactored system are easily affected by various factors, making it difficult to maintain their stoichiometric balance. An alternative approach is to reassemble the system as polycistronic modules, which maintain protein complex stoichiometry. However, large polycistronic operons cannot easily be utilized to express bacterial CBS in eukaryotic cells.

Thus, there is still an essential requirement to express the complex biological system in host cells, especially in eukaryotic cells, and to maintain relative expression levels (stoichiometry) of the multiple encoded protein components.

### SUMMARY OF THE INVENTION

The inventors solved the above technical problems by grouping the components of the complex biological system according to their natural expression levels and constructing fusion expression vectors for each group of genes. Each fusion expression vector constructed expresses a single polyprotein in the cells, which is then cleaved by proteases and releases multiple functional components of the complex biological system. The above method is capable of simplifying the expression procedure of complex biological systems in host cells, reduce the number of vectors that need to be transformed, and maintain the natural stoichiometry between their various components. The method of the present invention makes it feasible to exogenously express a complex biological system with a corresponding function in a host cell, particularly in a eukaryotic cell.

Accordingly, in one aspect, the invention relates to a method for expressing a complex biological system comprising multiple genes encoding multiple components in a host cell, the method comprising:

a) determining the expression level of each gene in its native operon location;

b) grouping said genes according to the expression level of each gene determined in a), wherein each group comprises genes with similar expression levels;

c) constructing a fusion expression vector for each group of genes according to the grouping in b), wherein the fusion expression vector comprises coding sequences of all genes of its corresponding group, and wherein the coding sequences are directly linked in-frame, linked via a nucleotide sequence encoding a linker, or separated by a nucleotide sequence encoding a cleavage sequence recognized by a protease, thus obtaining a set of fusion expression vectors;

d) introducing the set of fusion expression vectors into a host cell to express a polyprotein from each expression vector;

e) expressing the protease in the host cell to cleave the polyproteins, wherein components encoded by coding sequences directly linked or linked via a nucleotide sequence encoding a linker are expressed as a fusion protein, and wherein components encoded by coding sequences separated by a nucleotide sequence encoding the cleavage sequence are released after protease cleavage.

In some embodiments, “having similar expression levels” means that the expression level of any of the genes is not more than 10 times of that of other genes, preferably the expression level of any of the genes is not more than 5 times of that of other genes, more preferably the expression level of any of the genes is not more than 3 times of that of other genes, and even more preferably the expression level of any of the genes is not more than 2 times of that of other genes.

In some embodiments of above methods, step c) further comprises testing the activity of the components encoded by genes in each group when expressed as a fusion protein, wherein coding sequences of two or more components that are capable of maintaining the activity of each component when expressed as fusion proteins are directly linked in-frame, or linked via a nucleotide sequence encoding a linker, and other coding sequences are separated by a nucleotide sequence encoding a cleavage sequence recognized by a protease.

In some embodiments, being capable of maintaining the activity of each component when expressed as fusion proteins means that when expressed as a fusion protein, the activity of each component is at least 30%, at least 35%, at least 40%, at least 45%, at least 50%, at least 55%, at least 60%, at least 65%, at least 70%, at least 75%, at least 80%, at least 85%, at least 90%, at least 95% or 100% of its activity when expressed as a single protein. In some embodiments, being capable of maintaining the activity of each component when expressed as fusion proteins means that when expressed as a fusion protein, the activity of each component is at least 50%, at least 60%, or at least 70% of its activity when expressed as a single protein. In some embodiments, the activity is an enzymatic activity.

In some embodiments of above methods, step c) further comprises a step of arranging coding sequences in a construct, the step comprising testing each component for its tolerance in the presence of a residual sequence at the N-terminal or C-terminal after protease cleavage, wherein for a component with low tolerance in the presence of a residual sequence at the N-terminal, its coding sequence is arranged upstream of the coding sequences of other components; for a component with low tolerance in the presence of a residual sequence at the C-terminal, its coding sequence is arranged downstream of the coding sequences of other components; when there are two or more components with low tolerance in the presence of a residual sequence at the N-terminal in one group, only one of them is retained and its coding sequence is arranged upstream of the coding sequences, and other components with low tolerance in the presence of a residual sequence at the N-terminal are grouped into other groups; when there are two or more components with low tolerance in the presence of a residual sequence at the C-terminal in one group, only one of them is retained and its coding sequence is arranged downstream of the coding sequences, and other components with low tolerance in the presence of a residual sequence at the C-terminal are grouped into other groups.

In some embodiments, a component with low tolerance in the presence of a residual sequence at the N-terminal or C-terminal is defined as that the activity of the component is reduced by at least 10%, at least 20%, at least 30%, at least 40%, at least 50%, at least 60%, at least 70%, at least 80%, or at least 90% in the presence of a residual sequence at its N-terminal or C-terminal. In some embodiments, a component with low tolerance in the presence of a residual sequence at the N-terminal or C-terminal is defined as: (activity in the presence of a residual sequence/activity in the absence of a residual sequence %)ⁿ is less than 30%, less than 40%, less than 50%, less than 60%, less than 70%, less than 80%, or less than 90%, wherein n is the number of genes of said complex biological system. In some embodiments, the activity is an enzymatic activity.

In any embodiment of above methods, genes originally with different expression levels may achieve similar expression levels by adjusting the copy number of the coding sequences and are grouped into one group. For example, in the case where the expression level of a first gene is about 2 times of that of a second gene, the copy number of the coding sequence of the second gene may be adjusted to 2 and the above first and second genes are grouped into the same group.

In any embodiment of above methods, for one or more fusion expression vectors, for example, each of the fusion expression vectors may use a native expression control sequence of one of genes in its corresponding group or another expression control sequence having a similar expression level therewith. Said another expression control sequence may be an expression control sequence from other genes, or a synthetic expression control sequence.

In some embodiments of above methods, the protease may be selected from the group consisting of thrombin, Factor Xa, enterokinase, Tobacco Etch Virus (TEV) protease, PreScission and HRV 3C protease. In some embodiments, the protease is TEV protease.

In some embodiments of above methods, the host cell is a prokaryotic cell or a eukaryotic cell. For example, the prokaryotic cell may be selected from Pseudomonas fluorescens, Bacillus subtilis, Pseudomonas protegens, Pseudomonas putida, Pseudomonas veronii, Pseudomonas taetrolens, Pseudomonas balearica, Pseudomonas stutzeri, Pseudomonas aeruginosa, Pseudomonas syringae, Bacillus amyloliquefaciens, Burkholderia phytofirmans, Gluconacetobacter diazotrophicus, Herbaspirillum seropedicae, Bacillus cereus. For example, the eukaryotic cell may be selected from the cell of following species: Oryza sativa, Triticum aestivum, Zea mays, Sorghum bicolor, Setaria italica, Solanum tuberosum, Ipomoea batatas, Arachis hypogaea, Brassica napus, Malva farviflora, Sesamum indicum, Olea europaea, Elaeis guineensis, Saccharum officinarum, Beta vulgaris, Gossypium spp.

In some embodiments, the method of the invention may be used to express a complex biological system selected from the group consisting of alkane degradation pathway, nitrogen fixation system, polychlorinated biphenyl degradation system, bioplastic biosynthetic system (poly(3-hydroxybutryrate) biosynthetic system), nonribosomal peptide biosynthetic system, polyketide biosynthetic system, terpenoid biosynthetic system, oligosaccharide biosynthetic system, indolocarbazole biosynthetic system.

In some embodiments, the complex biological system is a nitrogen fixation system. In some embodiments, the nitrogen fixation system comprises the following genes: nifH, nifD, nifK, nifY, nifE, nifN, nifX, nifB, nifU, nifU, nifS, nifV, nifM, nifJ, nifF and optionally nifT, nifX, nifQ, nifW, nifZ. In some embodiments, the nitrogen fixation system is from Klebsiella oxytoca.

In any embodiment of above methods, the genes are grouped into three to seven groups, for example, three groups, four groups, five groups, six groups or seven groups. In some embodiments, the genes are grouped into four groups, five groups or six groups.

In some embodiments of above methods of expressing a nitrogen fixation system, the following genes are grouped into one group: nifH, nifD, nifK. In some embodiments, nifH, nifD, nifK genes are grouped into one group and the corresponding fusion expression vector has the following manner of arrangement and connection from upstream to downstream: nifH-cleav-nifD-cleav-nifK, wherein cleav is a nucleotide sequence encoding a cleavage sequence recognized by a protease.

In some embodiments, the following genes are grouped into one group: nifE, nifN, nifB. In some embodiments, nifE, nifN, nifB genes are grouped into one group and the corresponding fusion expression vector has the following manner of arrangement and connection from upstream to downstream: nifE-cleav-nifN-linker-nifB, wherein cleav is a nucleotide sequence encoding a cleavage sequence recognized by a protease, and linker is a nucleotide sequence encoding a linker. In a preferable embodiment, the linker is (GGGGS)m, wherein m is an integer from 1-10. For example, the linker may be (GGGGS)₅.

In some embodiments, the following genes are grouped into one group: nifF, nifM, nifY. In some embodiments, nifF, nifM, nifY genes are grouped into one group and the corresponding fusion expression vector has the following manner of arrangement and connection from upstream to downstream: nifF-cleav-nifM-cleav-nifY, wherein cleav is a nucleotide sequence encoding a cleavage sequence recognized by a protease.

In some embodiments, the following genes are grouped into one group: nifJ, nifV and optionally nifW, nifZ. In some embodiments, the fusion expression vector corresponding to the above gene grouping has the following structure from upstream to downstream: nifJ-cleav-nifV-cleav-nifW, nifJ-cleav-nifV-cleav-nifZ, or nifJ-cleav-nifV-cleav-nifW-cleav-nifZ, wherein cleav is a nucleotide sequence encoding a cleavage sequence recognized by a protease.

In any embodiment of above methods, nifU and nifS genes are grouped into one group, or nifU and nifS are expressed as separate genes. In an embodiment in which nifU and nifS genes are grouped into one group, the fusion expression vector comprising the coding sequences of nifU and nifS genes may have the following manner of arrangement and connection from upstream to downstream: nifU-cleav-nifS, wherein cleav is a nucleotide sequence encoding a cleavage sequence recognized by a protease.

In a further embodiment, the coding sequences of nifH, nifD, nifK, nifY, nifE, nifN, nifB, nifU, nifS, nifV, nifM, nifJ, nifF and optionally nifW, nifZ genes of a nitrogen fixation system are cloned into five fusion expression vectors in the following manner of arrangement and connection:

a) nifH-cleav-nifD-cleav-nifK;

b) nifE-cleav-nifN-linker-nifB;

c) nifU-cleav-nifS;

d) nifJ-cleav-nifV-cleav-nifW, or nifJ-cleav-nifV-cleav-nifZ; and

e) nifF-cleav-nifM-cleav-nifY,

wherein cleav is a nucleotide sequence encoding a cleavage sequence recognized by a protease, and linker is a nucleotide sequence encoding a linker.

In some other embodiments, the coding sequences of nifH, nifD, nifK, nifY, nifE, nifN, nifB, nifU, nifS, nifV, nifM, nifJ, nifF and nifW genes of a nitrogen fixation system are cloned into six fusion expression vectors in the following manner of arrangement and connection:

a) nifH-cleav-nifD-cleav-nifK;

b) nifE-cleav-nifN-linker-nifB;

c) nifU;

d) nifS;

e) nifJ-cleav-nifV-cleav-nifW; and

f) nifF-cleav-nifM-cleav-nifY,

wherein cleav is a nucleotide sequence encoding a cleavage sequence recognized by a protease, and linker is a nucleotide sequence encoding a linker.

In another aspect, the invention relates to a vector comprising coding sequences of two or more genes of a complex biological system, said complex biological system comprise multiple genes encoding multiple components, said two or more genes have similar expression levels in their native operon locations, wherein the coding sequences of the two or more genes are directly linked in-frame, linked via a nucleotide sequence encoding a linker, or separated by a nucleotide sequence encoding a cleavage sequence recognized by a protease.

In some embodiments, the vector is an expression vector, such as a fusion expression vector. In other embodiments, the vector is a cloning vector.

In some embodiments, in said vector, coding sequences of two or more components that are capable of maintaining the activity of each component when expressed as fusion proteins are directly linked in-frame, or linked via a nucleotide sequence encoding a linker, and other coding sequences are separated by a nucleotide sequence encoding a cleavage sequence recognized by a protease. In some embodiments, being capable of maintaining the activity of each component when expressed as fusion proteins means that when expressed as a fusion protein, the activity of each component is at least 30%, at least 35%, at least 40%, at least 45%, at least 50%, at least 55%, at least 60%, at least 65%, at least 70%, at least 75%, at least 80%, at least 85%, at least 90%, at least 95% or 100% of its activity when expressed as a single protein. In some embodiments, being capable of maintaining the activity of each component when expressed as a fusion protein means that when expressed as a fusion protein, the activity of each component is at least 50%, at least 60%, or at least 70% of its activity when expressed as a single protein. In some embodiments, the activity is an enzymatic activity.

In some embodiments of above vectors, the coding sequence of a component with low tolerance in the presence of a residual sequence at the N-terminal after protease cleavage is arranged upstream of the coding sequences of other components; the coding sequence of a component with low tolerance in the presence of a residual sequence at the C-terminal is arranged downstream of the coding sequences of other components. In some embodiments, the component with low tolerance in the presence of a residual sequence at the N-terminal or C-terminal is defined as that the activity of the component is reduced by at least 10%, at least 20%, at least 30%, at least 40%, at least 50%, at least 60%, at least 70%, at least 80%, or at least 90% in the presence of a residual sequence at its N-terminal or C-terminal. In some embodiments, the component with low tolerance in the presence of a residual sequence at the N-terminal or C-terminal is defined as: (activity in the presence of residual sequences/activity in the absence of residual sequences %)ⁿ is less than 30%, less than 40%, less than 50%, less than 60%, less than 70%, less than 80%, or less than 90%, wherein n is the number of genes of said complex biological system. In some embodiments, the activity is an enzymatic activity.

In any embodiment of above vectors, the vector comprises different copy numbers of coding sequences for the two or more genes, so that genes originally with different expression levels achieve similar expression levels. For example, in the case where the expression level of a first gene is about 2 times that of a second gene, the copy number of the coding sequence of the second gene may be adjusted to 2 and the above first and second genes are grouped into the same group.

In any embodiment of above vectors, in particular in the case where the vector is an expression vector, the vector may have a native expression control sequence of one of the two or more genes or another expression control sequence having a similar expression level therewith. Said another expression control sequence may be an expression control sequence from other genes, or a synthetic expression control sequence.

In any embodiment of above vectors, the protease may be selected from the group consisting of thrombin, Factor Xa, enterokinase, Tobacco Etch Virus (TEV) protease, PreScission and HRV 3C protease. In some embodiments, the protease is TEV protease.

In some embodiments, the vector is a fusion expression vector for expression in a host cell, and the host cell may be a prokaryotic cell or a eukaryotic cell. In some embodiments, the prokaryotic cell may be selected from: Pseudomonas fluorescens, Bacillus subtilis, Pseudomonas protegens, Pseudomonas putida, Pseudomonas veronii, Pseudomonas taetrolens, Pseudomonas balearica, Pseudomonas stutzeri, Pseudomonas aeruginosa, Pseudomonas syringae, Bacillus amyloliquefaciens, Burkholderia phytofirmans, Gluconacetobacter diazotrophicus, Herbaspirillum seropedicae, Bacillus cereus. In some embodiments, the eukaryotic cell may be a cell selected from the following species: Oryza sativa, Triticum aestivum, Zea mays, Sorghum bicolor, Setaria italica, Solanum tuberosum, Ipomoea batatas, Arachis hypogaea, Brassica napus, Malva farviflora, Sesamum indicum, Olea europaea, Elaeis guineensis, Saccharum officinarum, Beta vulgaris, Gossypium spp.

In any embodiment of above vectors, the complex biological system may be selected from alkane degradation pathway, nitrogen fixation system, polychlorinated biphenyl degradation system, bioplastic biosynthetic system (poly(3-hydroxybutryrate) biosynthetic system), nonribosomal peptide biosynthetic system, polyketide biosynthetic system, terpenoid biosynthetic system, oligosaccharide biosynthetic system, indolocarbazole biosynthetic system.

In some embodiments, the complex biological system is a nitrogen fixation system.

In some embodiments, the nitrogen fixation system comprises the following genes: nifH, nifD, nifK, nifY, nifE, nifN, nifX, nifB, nifU, nifU, nifS, nifV, nifM, nifJ, nifF and optionally nifT, nifX, nifQ, nifW, nifZ.

In some embodiments, the nitrogen fixation system is from Klebsiella oxytoca.

In some embodiments, the vector comprises coding sequences of the following genes: nifH, nifD, nifK. Preferably, the vector has the following manner of arrangement and connection from upstream to downstream: nifH-cleav-nifD-cleav-nifK, wherein cleav is a nucleotide sequence encoding a cleavage sequence recognized by a protease.

In some embodiments, the vector comprises coding sequences of the following genes: nifE, nifN, nifB. Preferably, the vector has the following manner of arrangement and connection from upstream to downstream: nifE-cleav-nifN-linker-nifB, wherein cleav is a nucleotide sequence encoding a cleavage sequence recognized by a protease, and linker is a nucleotide sequence encoding a linker. In some embodiments, the linker is (GGGGS)m, wherein m is an integer from 1-10. For example, the linker may be (GGGGS)₅.

In some embodiments, the vector comprises coding sequences of the following genes: nifF, nifM, nifY. Preferably, the vector has the following manner of arrangement and connection from upstream to downstream: nifF-cleav-nifM-cleav-nifY, wherein cleav is a nucleotide sequence encoding a cleavage sequence recognized by a protease.

In some embodiments, the vector comprises coding sequences of the following genes: nifJ, nifV and optionally nifW, nifZ. Preferably, the vector has the following manner of arrangement and connection from upstream to downstream: nifJ-cleav-nifV-cleav-nifW, nifJ-cleav-nifV-cleav-nifZ, or nifJ-cleav-nifV-cleav-nifW-cleav-nifZ, wherein cleav is a nucleotide sequence encoding a cleavage sequence recognized by a protease.

In some embodiments, the vector comprises coding sequences of the following genes: nifU, nifS. Preferably, the vector has the following manner of arrangement and connection from upstream to downstream: nifU-cleav-nifS, wherein cleav is a nucleotide sequence encoding a cleavage sequence recognized by a protease.

In yet another aspect, the invention relates to a vector composition comprising multiple vectors each comprising a coding sequence of one or more genes of a complex biological system, said complex biological system comprising multiple genes encoding multiple components, wherein the coding sequence of each gene of the complex biological system is present in one of the vectors, and the multiple vectors collectively comprise coding sequences of all genes of the complex biological system, wherein in a vector comprising coding sequences of two or more genes, said two or more genes have similar expression levels in their native operon locations, wherein the coding sequences of the two or more genes are directly linked in-frame, linked via a nucleotide sequence encoding a linker, or separated by a nucleotide sequence encoding a cleavage sequence recognized by a protease.

In some embodiments of the vector composition, in a vector comprising coding sequences of two or more genes, coding sequences of genes of two or more components that are capable of maintaining the activity of each component when expressed as fusion proteins are directly linked in-frame, or linked via a nucleotide sequence encoding a linker, and other components are separated by a nucleotide sequence encoding a cleavage sequence recognized by a protease.

In some embodiments, being capable of maintaining the activity of each component when expressed as fusion proteins means that when expressed as a fusion protein, the activity of each component is at least 30%, at least 35%, at least 40%, at least 45%, at least 50%, at least 55%, at least 60%, at least 65%, at least 70%, at least 75%, at least 80%, at least 85%, at least 90%, at least 95% or 100% of its activity when expressed as a single protein. In some embodiments, being capable of maintaining the activity of each component when expressed as a fusion protein means that when expressed as a fusion protein, the activity of each component is at least 50%, at least 60%, or at least 70% of its activity when expressed as a single protein. In some embodiments, the activity is an enzymatic activity.

In some embodiments of the vector composition, in a vector comprising coding sequences of two or more genes, the coding sequence of a component with low tolerance in the presence of a residual sequence at the N-terminal after protease cleavage is arranged upstream of the coding sequences of other components; the coding sequences of a component with low tolerance in the presence of a residual sequence at the C-terminal is arranged downstream of the coding sequences of other components. In some embodiments, the component with low tolerance in the presence of a residual sequence at the N-terminal or C-terminal is defined as that the activity of the component is reduced by at least 10%, at least 20%, at least 30%, at least 40%, at least 50%, at least 60%, at least 70%, at least 80%, or at least 90% in the presence of a residual sequence at its N-terminal or C-terminal. In some embodiments, the component with low tolerance in the presence of a residual sequence at the N-terminal or C-terminal is defined as: (activity in the presence of a residual sequence/activity in the absence of a residual sequence %)ⁿ is less than 30%, less than 40%, less than 50%, less than 60%, less than 70%, less than 80%, or less than 90%, wherein n is the number of genes of said complex biological system. In some embodiments, the activity is an enzymatic activity.

In some embodiments of the vector composition, in a vector comprising coding sequences of two or more genes, genes originally with different expression levels achieve similar expression levels by including different copy numbers of the coding sequences.

In some embodiments, one or more vectors in the vector composition, for example, each of the vectors has a native expression control sequence of one of the coding sequences of the one or more genes comprised therein or an expression control sequence having a similar expression level therewith.

In any embodiment of the vector compositions, the protease may be selected from the group consisting of thrombin, Factor Xa, enterokinase, Tobacco Etch Virus (TEV) protease, PreScission and HRV 3C protease. In some embodiments, the protease is TEV protease.

In some embodiments, one or more vectors in the vector composition, for example, each of the vectors, is a fusion expression vector for expression in a host cell. In some embodiments, the host cell is a prokaryotic cell or a eukaryotic cell. In some embodiments, the prokaryotic cell may be selected from: Pseudomonas fluorescens, Bacillus subtilis, Pseudomonas protegens, Pseudomonas putida, Pseudomonas veronii, Pseudomonas taetrolens, Pseudomonas balearica, Pseudomonas stutzeri, Pseudomonas aeruginosa, Pseudomonas syringae, Bacillus amyloliquefaciens, Burkholderia phytofirmans, Gluconacetobacter diazotrophicus, Herbaspirillum seropedicae, Bacillus cereus. In some embodiments, the eukaryotic cell may be a cell selected from the following species: Oryza sativa, Triticum aestivum, Zea mays, Sorghum bicolor, Setaria italica, Solanum tuberosum, Ipomoea batatas, Arachis hypogaea, Brassica napus, Malva farviflora, Sesamum indicum, Olea europaea, Elaeis guineensis, Saccharum officinarum, Beta vulgaris, Gossypium spp.

In any embodiment of the vector compositions, the complex biological system may be selected from: alkane degradation pathway, nitrogen fixation system, polychlorinated biphenyl degradation system, bioplastic biosynthetic system (poly(3-hydroxybutryrate) biosynthetic system), nonribosomal peptide biosynthetic system, polyketide biosynthetic system, terpenoid biosynthetic system, oligosaccharide biosynthetic system, indolocarbazole biosynthetic system.

In some embodiments, the complex biological system is a nitrogen fixation system.

In some embodiments, the nitrogen fixation system comprises the following genes: nifH, nifD, nifK, nifY, nifE, nifN, nifX, nifB, nifU, nifU, nifS, nifV, nifM, nifJ, nifF and optionally nifT, nifX, nifQ, nifW, nifZ.

In some embodiments, the nitrogen fixation system is from Klebsiella oxytoca.

In any embodiment of the vector compositions, the vector composition may comprise three to seven vectors, for example three, four, five, six or seven vectors. In some embodiments, the vector composition comprises four, five or six vectors.

In some embodiments, the vector composition comprises a vector comprising coding sequences of the following genes: nifH, nifD, nifK. Preferably, the vector has the following manner of arrangement and connection from upstream to downstream: nifH-cleav-nifD-cleav-nifK, wherein cleav is a nucleotide sequence encoding a cleavage sequence recognized by a protease.

In some embodiments, the vector composition comprises a vector comprising coding sequences of the following genes: nifE, nifN, nifB. Preferably, the vector has the following manner of arrangement and connection from upstream to downstream: nifE-cleav-nifN-linker-nifB, wherein cleav is a nucleotide sequence encoding a cleavage sequence recognized by a protease, and linker is a nucleotide sequence encoding a linker. In some embodiments, the linker is (GGGGS)m, wherein m is an integer from 1-10.

In some embodiments, the vector composition comprises a vector comprising coding sequences of the following genes: nifF, nifM, nifY. Preferably, the vector has the following manner of arrangement and connection from upstream to downstream: nifF-cleav-nifM-cleav-nifY, wherein cleav is a nucleotide sequence encoding a cleavage sequence recognized by a protease.

In some embodiments, the vector composition comprises a vector comprising coding sequences of the following genes: nifJ, nifV and optionally nifW, nifZ. Preferably, the vector has the following manner of arrangement and connection from upstream to downstream: nifJ-cleav-nifV-cleav-nifW, nifJ-cleav-nifV-cleav-nifZ, or nifJ-cleav-nifV-cleav-nifW-cleav-nifZ, wherein cleav is a nucleotide sequence encoding a cleavage sequence recognized by a protease.

In some embodiments, the vector composition comprises a vector comprising coding sequences of nifU and nifS genes, or comprises a vector comprising a coding sequence of nifU gene and a vector comprising a coding sequence of nifS gene. In case that the vector composition comprises a vector comprising coding sequences of nifU and nifS genes, the vector preferably has the following manner of arrangement and connection from upstream to downstream: nifU-cleav-nifS, wherein cleav is a nucleotide sequence encoding a cleavage sequence recognized by a protease.

In some embodiments of the vector compositions, the vector composition comprises the following vectors:

a) a vector with nifH-cleav-nifD-cleav-nifK;

b) a vector with nifE-cleav-nifN-linker-nifB;

c) a vector with nifU-cleav-nifS;

d) a vector with nifJ-cleav-nifV-cleav-nifW, or a vector with nifJ-cleav-nifV-cleav-nifZ; and

e) a vector with nifF-cleav-nifM-cleav-nifY, wherein cleav is a nucleotide sequence encoding a cleavage sequence recognized by a protease, and linker is a nucleotide sequence encoding a linker.

In other embodiments of the vector compositions, the vector composition comprises the following vectors:

a) a vector with nifH-cleav-nifD-cleav-nifK;

b) a vector with nifE-cleav-nifN-linker-nifB;

c) a vector with nifU;

d) a vector with nifS;

e) a vector with nifJ-cleav-nifV-cleav-nifW; and

f) a vector with nifF-cleav-nifM-cleav-nifY,

wherein cleav is a nucleotide sequence encoding a cleavage sequence recognized by a protease, and linker is a nucleotide sequence encoding a linker.

In any of the embodiments described above with respect to the vector composition, the vector composition may further comprises an expression vector of the coding sequence of the protease.

In one aspect, the invention relates to a host cell comprising a vector or a vector composition of the invention.

In another aspect, the invention relates to a method of transforming a host cell comprising a step of transducing or transfecting the host cell with a vector or a vector composition of the invention.

In yet another aspect, the invention relates to use of a vector or a vector composition of the invention for transforming a host cell.

In any of the embodiments described above with respect to the host cell, the method of transforming a host cell and the use of transforming a host cell, the host cell may be a prokaryotic cell or a eukaryotic cell. In some embodiments, the prokaryotic cell may be selected from: Pseudomonas fluorescens, Bacillus subtilis, Pseudomonas protegens, Pseudomonas putida, Pseudomonas veronii, Pseudomonas taetrolens, Pseudomonas balearica, Pseudomonas stutzeri, Pseudomonas aeruginosa, Pseudomonas syringae, Bacillus amyloliquefaciens, Burkholderia phytofirmans, Gluconacetobacter diazotrophicus, Herbaspirillum seropedicae, Bacillus cereus. In some embodiments, the eukaryotic cell may be a cell selected from the following species: Oryza sativa, Triticum aestivum, Zea mays, Sorghum bicolor, Setaria italica, Solanum tuberosum, Ipomoea batatas, Arachis hypogaea, Brassica napus, Malva farviflora, Sesamum indicum, Olea europaea, Elaeis guineensis, Saccharum officinarum, Beta vulgaris, Gossypium spp.

### DESCRIPTION OF THE DRAWINGS

FIG. 1 is a schematic diagram showing exemplary steps of the method of the present invention to express a complex biological system.

FIG. 2 is a graph showing the results of the relative nitrogenase activity of the products of fusion expression vectors with different manners of arrangement and connection after grouping the genes of the nitrogen fixation system using the method of the invention. Relative nitrogenase activity is shown in cases where TEVp is expressed or not expressed. FIG. 2A shows the results of the nitrogenase activity in different manners of arrangement and connection for the nifHDK group. FIG. 2B shows the results of the nitrogenase activity in different manners of arrangement and connection for the nifENB group. FIG. 2C shows the results of the nitrogenase activity in different manners of arrangement and connection for the nifUS group. FIG. 2D shows the results of the nitrogenase activity in different manners of arrangement and connection for the nifFMY group. FIG. 2E shows the results of the nitrogenase activity in different manners of arrangement and connection for the nifJV group and optionally nifWZ group.

FIG. 3 is a graph showing the results of the overall relative activity of the nitrogen fixation system when the genes of the nitrogen fixation system are grouped using the method of the present invention, and the set of fusion expression vectors constructed in different manners of arrangement are expressed in host cells. The acetylene reduction assay and ¹⁵N assimilation assay were used to show relative nitrogenase activity.

FIG. 4 shows a photograph of E. coli grown on a solid medium using N₂ as the sole nitrogen source after transfection of E. coli with the fusion expression vectors using grouping and arrangement manner VIII shown in FIG. 3.

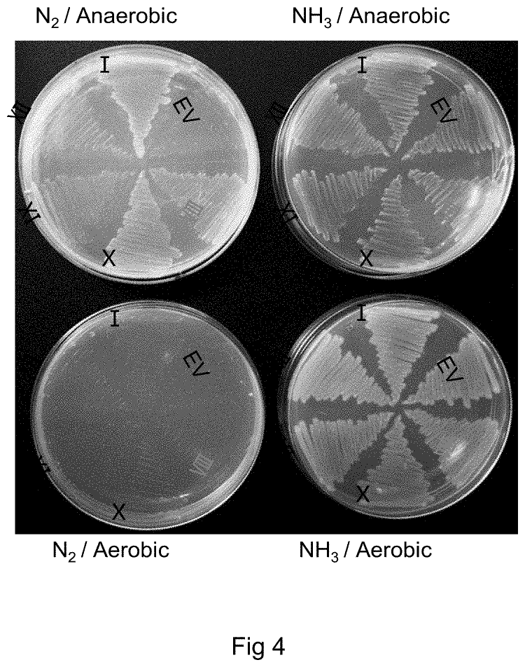

FIG. 5 shows a graph of the results of expressing nifUS polyprotein in yeast mitochondria of eukaryotic host cells using the grouping and polyprotein-based expression strategy of the invention. FIG. 5A shows a schematic of each vector constructed. FIGS. 5B and 5C show the results of Western blotting for the corresponding expression products.

### DETAILED DESCRIPTION OF THE INVENTION

### Terms and Definitions

Unless otherwise defined herein, scientific and technical terms used in combination with the present application shall have meanings as commonly understood by those of ordinary skill in the art to which this disclosure belongs. The terms used herein are for the purpose of describing particular embodiments only and are not intended to limit the scope of the invention.

As used herein, the term “nucleic acid” or “polynucleotide” refers to oligomers and polymers of any length consisting essentially of nucleotides, such as deoxyribonucleotides and/or ribonucleotides. Nucleic acids may comprise purine and/or pyrimidine bases and/or other natural (e.g. xanthine, inosine, hypoxanthine), chemically or biochemically modified (e.g. methylation), unnatural or derived nucleotide bases. The backbone of a nucleic acid may comprise sugars and phosphate groups that are typically found in RNA or DNA, and/or one or more modified or substituted sugars and/or one or more modified or substituted phosphate groups. Modifications of phosphate groups or sugars may be introduced to improve stability, resistance to enzymatic degradation, or some other useful properties. A “nucleic acid” may be, for example, double-stranded, partially double-stranded, or single-stranded. As single-stranded nucleic acid, the nucleic acid may be the sense or antisense strand. A “nucleic acid” may be circular or linear. As used herein, the term “nucleic acid” encompasses DNA and RNA, including genomes, pre-mRNA, mRNA, cDNA, recombinant or synthetic nucleic acids including vectors.

When referring to a nucleic acid in a recombinant host, “recombinant nucleic acid” means that at least a portion of the nucleic acid does not naturally occur in the same genomic location of the host cell. For example, a recombinant nucleic acid may comprise a coding sequence naturally occurring in a host cell under the control of a heterologous expression control sequence, or it may be an additional copy of a gene naturally occurring in the host cell, or the recombinant nucleic acid may comprise a heterologous coding sequence under the control of an endogenous expression control sequence.

The terms “protein” and “polypeptide” are used interchangeably herein and generally refer to polymers of amino acid residues linked by peptide bonds and do not limit the minimum length of the products. Thus, the above terms include peptides, oligopeptides, polypeptides, dimers (heterologous and homologous), multimers (heterologous and homologous), and the like. “Protein” and “polypeptide” encompass full-length proteins and fragments thereof. The term also includes post-expression modifications of the polypeptide, such as glycosylation, acetylation, phosphorylation, and the like. In addition, for the purposes of the present invention, “protein” and “polypeptide” also refer to variants obtained after modification, such as deletion, addition, insertion, and substitution (such as conservative amino acid substitutions), of the amino acid sequence of a natural protein or polypeptide.

For example, proteins and polypeptides may refer to variants of natural proteins or polypeptides that have at least 80%, at least 85%, at least 90%, at least 95%, at least 98%, or at least 99% sequence identity to natural proteins or polypeptides, provided that the variant retains the original function or activity of the natural protein or polypeptide.

The correlation between two amino acid sequences or between two nucleotide sequences can be described by the parameter “sequence identity”. The percentage of sequence identity between two sequences can be determined, for example, using a mathematical algorithm.

The percentage of sequence identity between two sequences can be determined, for example, using a mathematical algorithm. Non-limiting examples of such mathematical algorithms include the algorithm of Myers and Miller (1988) CABIOS 4:11-17, the local homology algorithm of Smith et al. (1981) Adv. Appl. Math. 2:482, homology alignment algorithm of Needleman and Wunsch (1970) J. Mol. Biol. 48:443-453, the method for searching homology of Pearson and Lipman (1988) Proc. Natl. Acad. Sci. 85:2444-2448, a modified version of the algorithm of Karlin and Altschul (1990) Proc. Natl. Acad. Sci. USA 87:2264 and the algorithm described in Karlin and Altschul (1993) Proc. Natl. Acad. Sci. USA 90:5873-5877. By using a program based on such a mathematical algorithm, sequence comparisons (i.e., alignments) for determining sequence identity can be performed. The program can be appropriately executed by a computer. Examples of such programs include, but are not limited to, CLUSTAL of the PC/Gene program, ALIGN program (Version 2.0), and GAP, BESTFIT, BLAST, FASTA, and TFASTA of the Wisconsin Genetics software package. Alignment using these programs may be performed, for example, by using initial parameters.

A “conservative amino acid substitution” refers to a substitution between amino acid residues having similar charge properties or side chain groups, which generally does not affect the normal function of the protein or polypeptide. Examples of conservative amino acid substitutions include substitutions between Phe, Trp, and Tyr if the substitution site is an aromatic amino acid; substitutions between Leu, Ile, and Val if the substitution site is a hydrophobic amino acid; substitutions between Gln and Asn if the substitution site is a polar amino acid; substitutions between Lys, Arg and His if the substitution site is a basic amino acid; substitutions between Asp and Glu if the substitution site is an acidic amino acid; and substitutions between Ser and Thr if it is an amino acid with a hydroxyl group.

The term “coding sequence” means a polynucleotide encoding the amino acid sequence of a protein or polypeptide. The boundaries of a coding sequence are generally determined by an open reading frame, which begins with a start codon (such as ATG, GTG or TTG) and ends with a stop codon (such as TAA, TAG or TGA). The coding sequence may be derived from genomic DNA, or synthetic DNA, or a combination thereof.

Due to the degeneracy of the genetic code, several nucleic acids may encode polypeptides having the same amino acid sequence. For example, the codons GCA, GCC, GCG, and GCU all encode the amino acid alanine. Thus, at each position identified as an alanine by a codon, the codon may be replaced with any other codon encoding alanine without altering the encoded polypeptide. Those of ordinary skill in the art will recognize that codon in a nucleic acid (except for AUG, which is usually the only codon for methionine, and TGG, which is usually the only codon for tryptophan) may be modified without altering the amino acid sequence of the protein or polypeptide it encodes. Therefore, a codon preference table suitable for the target host cell may be used to modify the codons in the coding sequence of the protein to obtain optimal expression in a particular host cell, such as a prokaryotic cell or a eukaryotic cell. Codon preferences in various hosts are known in the art.

The term “linked in-frame” refers to a nucleotide sequence, such as a coding sequence, linked or fused in a manner that does not change the normal trinucleotide reading frame (which encodes a single amino acid as a genetic codon) of the linked or fused coding sequence, that is, the above manner of connection does not change the amino acid sequence encoded by the coding sequence.

The term “expression control sequence” means a nucleic acid sequence necessary for expression of a polynucleotide encoding a mature polypeptide. Each expression control sequence may be native (i.e., from the same gene) or foreign (i.e., from a different gene) to the polynucleotide encoding the polypeptide, or native or foreign with respect to each other. Such expression control sequences include, but are not limited to, a leader, a polyadenylation sequence, a propeptide sequence, a promoter, a signal peptide sequence, and a transcription terminator. The expression control sequence includes at least a promoter, and transcription and translation termination signals. In some embodiments, an expression control sequence will increase the expression of a gene. In other embodiments, the expression control sequence will reduce the expression of the gene.

Promoters may be constitutive or inducible. Examples of constitutive promoters include, but are not limited to, the retrovirus Rous sarcoma virus (RSV) LTR promoter, cytomegalovirus (CMV) promoter, SV40 promoter, dihydrofolate reductase promoter, β-actin promoter, phosphoglycerate kinase (PGK) promoter, and EF1α promoter.

Inducible promoters allow the regulation of gene expression and can regulate gene expression by, for example, exogenous addition of compounds, environmental factors such as temperature, or specific physiological states, specific differentiation states of cells, and division cycles. Inducible promoters may be obtained from a variety of commercial sources. Those skilled in the art can also select other inducible promoters and systems as required. Examples of inducible promoters regulated by exogenous addition of compounds include, but are not limited to: zinc-induced goat metallothionein (MT) promoter, dexamethasone (Dex)-induced mouse mammary tumor virus (MMTV) promoter, T7 polymerase promoter system, ecdysone insect promoter, tetracycline suppression system, tetracycline induction system, RU486 induction system and rapamycin induction system.

In addition, the promoter may be a promoter of cells commonly used in eukaryotic expression systems or a promoter used in prokaryotic expression systems. Examples of promoters used in eukaryotic expression systems include, but are not limited to, CMV promoter, SV40 promoter, PGK promoter, EF1α promoter, β-actin promoter, Ubc promoter (human ubiquitin C gene-derived promoter), CAG promoter (hybrid mammalian promoter), TRE promoter (tetracycline response element promoter), UAS promoter (Drosophila promoter with Gal4 binding site), Ac5 promoter (Drosophila actin 5c gene-derived insect promoter), CaMKIIa promoter (Ca2⁺/calmodulin-dependent protein kinase II promoter), GAL1 and GAL10 promoters (yeast bidirectional promoter), TEF promoter (yeast transcription elongation factor promoter), GDS promoter (glyceraldehyde-3-phosphate dehydrogenase-derived yeast promoter), ADH1 promoter (yeast alcohol dehydrogenase I promoter), CaMV35S promoter (cauliflower virus-derived plant promoter), Ubi Promoter (maize ubiquitin gene promoter), H1 promoter (human polymerase III-derived RNA promoter) and U6 promoter (human U6-derived small nuclear promoter).

Examples of promoters used in prokaryotic expression systems include, but are not limited to, T7 promoter (T7 phage-derived promoter), T7lac promoter (T7 phage-derived promoter plus lac operon), Sp6 promoter (Sp6 phage-derived promoter), araBAD promoter (arabinose metabolism operon-derived promoter), trp promoter (tryptophan operon-derived promoter), lac promoter (lac operon-derived promoter), Ptac promoter (a hybrid promoter of the lac promoter and the trp promoter), and pL promoter (Lambda phage-derived promoter).

The term “operably linked” refers to a configuration in which an expression control sequence is located in an appropriate location relative to a coding sequence of a polynucleotide such that the expression control sequence directs the expression of the coding sequence.

The term “expression” refers to the step of converting genetic information of a polynucleotide into RNA by catalytic transcription of an enzyme (such as RNA polymerase), and converting the above-mentioned genetic information into a protein or polypeptide by translating mRNA on the ribosome. As used herein, the term “expression” includes any step involving the production of a polypeptide, including, but not limited to, transcription, post-transcriptional modification, translation, post-translational modification, and secretion.

The term “vector” refers to a vector that can autonomously replicate in a host cell, which is preferably a multicopy vector. In addition, the vector usually has a marker such as an antibiotic resistance gene for selecting a transformant. In addition, the vector may have a promoter and/or a terminator for expressing the introduced gene. The vector may be, for example, a vector derived from a bacterial plasmid, a viral vector, a vector derived from a yeast plasmid, a vector derived from a phage, a cosmid, a phagemid, or the like.

The term “expression vector” refers to a vector that enables a target gene to be expressed in a cell, and is generally a linear or circular DNA molecule that includes a polynucleotide encoding a protein or polypeptide and is operably linked to an expression control sequence.

Nucleic acids, such as vectors or expression vectors, can be delivered to prokaryotic and eukaryotic cells by various methods known in the art. Methods for delivering nucleic acids into cells include, but are not limited to, various chemical, electrochemical and biological methods such as heat shock transformation, electroporation, transfection such as liposome-mediated transfection, DEAE-Dextran-mediated transfection or calcium phosphate transfection. In addition, a method such as treating a recipient cell with calcium chloride to increase its permeability to DNA, and a method of preparing competent cells from cells at a growth stage and then transforming with DNA can be used. A method in which DNA recipient cells are made into protoplasts or spheroplasts (which can easily take up recombinant DNA), and then the recombinant DNA is introduced into the DNA recipient cells can also be used. The transformation method is not particularly limited, and those skilled in the art can select a suitable transformation method according to, for example, the host cell used and the type of vector or expression vector to be transformed.

The term “host cell” means any cell type that is readily transformed, transfected, transduced, etc. with a nucleic acid construct or expression vector comprising a polynucleotide of the invention. The term “host cell” encompasses any offspring of a parent cell that is different from the parent cell due to mutations that occur during replication. Host cells may be isolated cells or cell lines grown in culture, or cells present in living tissues or organisms.

In the present invention, the host cell may be a prokaryotic cell or a eukaryotic cell. The prokaryotic host cell may be any Gram-positive or Gram-negative bacteria. The host cell may also be a eukaryote, such as a mammalian, insect, plant, or fungal cell. Examples of prokaryotic cells include, for example, Pseudomonas fluorescens, Bacillus subtilis, Pseudomonas protegens, Pseudomonas putida, Pseudomonas veronii, Pseudomonas taetrolens, Pseudomonas balearica, Pseudomonas stutzeri, Pseudomonas aeruginosa, Pseudomonas syringae, Bacillus amyloliquefaciens, Burkholderia phytofirmans, Gluconacetobacter diazotrophicus, Herbaspirillum seropedicae, Bacillus cereus, etc.

Examples of eukaryotic cells include, for example, a cell of the following species: Oryza sativa, Triticum aestivum, Zea mays, Sorghum bicolor, Setaria italica, Solanum tuberosum, Ipomoea batatas, Arachis hypogaea, Brassica napus, Malva farviflora, Sesamum indicum, Olea europaea, Elaeis guineensis, Saccharum officinarum, Beta vulgaris, Gossypium spp.

Complex Biological System

Alkane Degradation Pathway

Numerous marine and terrestrial bacteria have the ability to utilize hydrocarbons as a carbon and energy source. The genes involved in the use of alkanes constitute a complex biological system, i.e. the alkane degradation pathway. Petroleum is a chemically diverse substance and there are a range of enzymes and related pathways that break down different classes of molecules in petroleum. The alkane degradation system in P. putida includes alkB to alkS genes, specifically: alkB, alkF, alkG, alkH, alkJ, alkK, alkL, tnpAI, alkN, orf8, orf9, orf10, orf12, alkT and alkS gene. The alkane degradation system in P. putida is one of the most well-studied alkane degradation systems and is able to degrade medium-length alkanes. The metabolic pathway begins with an alkane hydroxylase (AlkB—a membrane-associated non-heme diiron monooxygenase), which converts the alkane to an alcohol. Often, strains contain multiple alkane hydroxylases to give it the ability to degrade different alkane substrates. Electrons are delivered to AlkB by two rubredoxins (AlkF and AlkG). The alcohol is then converted to acyl-CoA in three steps mediated by AlkHJK, at which point it then enters other metabolic pathways. Two additional proteins, AlkL and AlkN, encode an importer and chemotaxis sensory protein, respectively. AlkS acts as an alkane sensor and up-regulates gene expression.

The alkane degradation pathway occurs in many phylogenetically and taxonomically distinct bacteria, which has a lower G+C content than the overall genome and is flanked by transposon genes, which indicate frequent horizontal transfer.

Organisms with alkane degradation pathways have been used in a wide variety of industrial applications. This includes a variety of uses in environmental clean-up, such as biosensing and site evaluation, fermenter-based waste treatment, and refinery and tanker waste treatment. Organisms and related pathways have been identified that can break down nearly all of the components of petroleum, including benzene, ethylbenzene, trimethylbenzene, toluene, ethyltoluene, xylene, naphthalene, methylnapthalene, phenanthrene, C₆-C₈ alkanes, C₁₄-C₂₀ alkanes, branched alkanes, and cymene. In addition, alkane-degrading organisms can be used as biocatalysts to add value to petroleum products. For example, Alcanivorax has been engineered to direct the carbon flux from alkanes to the production of the bioplastic precursor such as poly(hydroxyalkanoate)(PHA). Another important use of the alkane degradation pathway is for microbial enhanced oil recovery (MEOR), where bacteria with alkane degradation pathways are introduced into oil wells to facilitate secondary recovery. The injection of oil-degrading organisms can increase recovery by reducing viscosity or secreting surfactants. MEOR has been tested and applied worldwide.

Nitrogen Fixation System

The availability of nitrogen limits the growth of many organisms. In agriculture, fixed nitrogen (combined nitrogen) is a critical component of fertilizer. Converting nitrogen (N₂) into a form that can enter metabolism such as ammonia is quite difficult. In industry, the Haber-Bosch process can chemically convert N₂ to ammonia using high temperatures, high pressures and an iron catalyst. In contrast, biological nitrogen fixation uses a complex enzyme to perform this reaction.

Only prokaryotes and some archaea have the ability to fix nitrogen. Generally, all of the genes for nitrogen fixation are encoded by a complex biological system. The most well-studied nitrogen fixation system is from K. pneumoniae, which consists of 20 genes, specifically including nifQ, nifB, nifA, nifL, nifF, nifM, nifZ, nifW, nifV, nifS, nifU, nifX, nifN, nifE, nifY, nifT, nifK, nifD, nifH and nifJ. These genes encode all of the necessary components for nitrogen fixation, including the nitrogenase, a metabolic pathway for the synthesis of metal co-factors, electron transporter, and a regulatory network. Nitrogenase consists of two core proteins (NifH and the NifDK complex) that participate in a reaction cycle. The reaction is an energy and redox intensive reaction, with the reaction formula N₂+8e⁻+16ATP→2NH₃+16ADP+16Pᵢ+H₂.

Each reaction cycle includes the transfer of 1 electron and the consumption of 2 ATP (the energy of which is used to accelerate electron transfer). It is implemented by a transient interaction between NifH, which receives an electron from a variety of sources, and NifDK, which contains the reaction center where N₂ binds and fixation occurs. The cycle of binding, electron transfer, and dissociation needs to be repeated 8 times to fix a single N₂ molecule. Nitrogenase is a slow enzyme and its reaction rate is limited by the dissociation step. Three co-factors form the core of the electron transfer and catalysis: [Fe₄—S₄] in NifH, the P-cluster [Fe₈—S₇] in NifDK, and FeMo-co [Mo—Fe₇—S₉—X] where the reaction occurs. The enzymes involved in the synthesis of these co-factors and chaperones make up the majority of the genes of a nitrogen fixation system. NifF and NifJ are flavodoxins that use an electron source such as pyruvate to transfer electrons to NifH protein. Nitrogenase is extremely oxygen sensitive and expensive for the cells to make and run. A simple regulatory cascade is formed by the activator NifA and the anti-activator NifL, which integrate signals to ensure that the genes of the nitrogen fixation system are only expressed in the absence of oxygen and fixed nitrogen.

Since the earliest tools in genetic engineering were developed, it has been one of the goals of biologists to create cereal crops that can fix their own nitrogen. The complexity of the nitrogen fixation pathway and a lack of efficient tools for modifying non-model plants have hindered progress in this area. Although the entire gene of K. pneumoniae has been functionally transferred into E. coli, it is quite difficult to transform it into other organisms, especially eukaryotic cells, to perform its function, because it needs to transform a large number of genes while maintaining the balance of expression between individual genes.

Polychlorinated Biphenyl Degradation System

Some bacteria can use harmful organic pollutants as their sole source of carbon and energy. For example, Burkholderia xenovorans LB400 can subsist on polychlorinated biphenyls (PCBs), which are used widely as fire retardants and plasticizers in industry. The polychlorinated biphenyl degradation system of Burkholderia xenovorans LB400 includes the following genes: bphD, bphI, bphJ, bphH, bphK, bphC, bphB, bphA4, bphA3, bph1195, bphA2, bphA1 and bph1198. The capability for degradation of polychlorinated biphenyls has allowed Burkholderia xenovorans and other PCB metabolizing bacteria to be used for bioremediation of chemical spills. Highly chlorinated PCBs are reductively dehalogenated by organisms such as Dehalococcoides, which can use PCBs as a terminal electron acceptor for anaerobic respiration. And lower chlorinated PCBs are the substrate for the Burkholderia xenovorans degradation pathway, which consists of a series of enzyme-mediated oxidations culminating in the cleavage of one of the linked aromatic rings by the ring-opening dioxygenase BphC. The cleaved ring is converted to two equivalents of acetate in a three-step pathway, while the uncleaved ring is released as benzoic acid and then further processed to catechol by the protein products of the benABCD genes.

Several strategies are being employed to increase the number of PCBs that can be degraded microbially. Future efforts may attempt to introduce PCB degradation system into bacterial strains that synthesize compounds of industrial value, which would allow these strains to consume PCBs as feedstocks that would otherwise require expensive and environmentally unfriendly disposal.

Bioplastic Biosynthetic System

Many bacteria synthesize poly(3-hydroxybutyrate) (PHB) and other poly(hydroxyalkanoates) (PHAs) as a means of storing carbon and energy intracellularly. The bioplastic biosynthetic system, exemplified by the phbC1, phbA, phbB1 and phbR genes in Ralstonia eutropha, catalyzes a pathway consisting of three steps: PhbA catalyzes a Claisen condensation to convert two molecules of acetyl-CoA to acetoacetyl-CoA, PhbB reduces acetoacetyl-CoA to 3-hydroxybutryl-CoA, and PhbC polymerizes 3-hydroxybutryl-CoA with release of CoA to form PHB. PHB is hydrophobic and accumulates in cytoplasmic granules.

PHB and other PHAs are versatile bioplastics. Biodegradable forms of a diverse set of products are produced from bacterially synthesized PHAs. Efforts to metabolically engineer the biosynthesis of bioplastics are proceeding along two tracks. In one aspect, the genes for the production of PHB and other PHAs have been introduced into plants in order to realize the benefits of using CO₂ as a carbon source rather than fermentation feedstocks. However, these efforts have been only modestly successful. To date, the best PHA production titer seen in plants is only ˜10% of dry weight. In the other aspect, engineering approaches including genetic engineering and the provision of unnatural substrate derivatives in the fermentation broth have led to the optimization of PHA yields in native and engineered hosts and the production of novel PHA derivatives.

Nonribosomal Peptide Biosynthetic System

Nonribosomal peptides (NRPs) are a class of peptidic small molecules that includes the antibiotic vancomycin, and the immunosuppressant cyclosporine and echinomycin, etc. Echinomycin, a DNA-damaging NRP from the quinoxaline class, the biosynthetic system of which includes ecm1-ecm18 genes (ecm1, ecm2, ecm3, ecm4, ecm5, ecm6, ecm7, ecm8, ecm9, ecm10, ecm11, ecm12, ecm13, ecm14, ecm15, ecm16, ecm17, ecm18), encodes four categories of gene products: (1) Genes for self-contained metabolic pathways that provide unusual monomers. Eight ecm-encoded enzymes convert tryptophan into quinoxaline-2-carboxylic acid (QC), an unusual monomer that enables echinomycin to intercalate between DNA base pairs: (2) Genes for an assembly-line-like enzyme known as an NRP synthetase (NRPS) that link monomers (typically amino acids) into a peptide and then release it from covalent linkage to the assembly line, often with concomitant macrocyclization. The ecm genes encode two NRPS enzymes, Ecm6 (2608 amino acids) and Ecm7 (3135 amino acids), that convert QC, serine, alanine, cysteine, and valine into a cyclic, dimeric decapeptidolactone; (3) Genes for chemical ‘tailoring’ after release from the NRPS. Two ecm-encoded enzymes oxidatively fuse the two cysteine sidechains into a thioacetal; and (4) Genes that encode regulatory and resistance functions. Transporters are also commonly found in NRP biosynthetic system.

There are two ways in which synthetic biology is being used in the area of NRPS engineering. In one aspect, efforts are being made to express NRPS biosynthetic system in heterologous hosts. Expression of NRPS biosynthetic system in a heterologous host can serve three purposes: making the encoded NRP accessible for structure elucidation or biological characterization, particularly useful if the native host is unknown or unculturable; making the genes easier to manipulate, which is useful if the native host is not amenable to genetics; and improving the production titer of its small molecule product, which is helpful if NRPS biosynthetic system is repressed by various regulatory systems in the native host. In the other aspect, engineering by replacing portions of NRPS genes with variants from other genes leads to the incorporation of alternative amino acid building blocks. This technique has been used most extensively to generate derivatives of the NRP antibiotic daptomycin.

Polyketide Biosynthetic System

Polyketides (PKs) are a class of acetate- and propionate-derived small molecules that includes the immunosuppressant FK506, the antibiotic tetracycline, the cholesterol-lowering agent lovastatin, and a number of rapamycin analogues made by genetic engineering. The biosynthetic pathways for PKs and fatty acids are similar in their chemical logic and use related enzymes: both involve the polymerization of acetate- or propionate-derived monomers by a series of Claisen condensations followed by reduction of the resulting β-ketothioester.

The biosynthetic system for erythromycin, an antibacterial PK from the macrolide class, including the following genes: ery0712, eryK, eryBVII, eryCV, eryCIV, eryBVI, eryCVI, eryBV, eryBIV, eryAI, ery0722, eryAII, eryAIII, eryCII, eryCIII, eryBII, eryG, ery0729, eryF, eryBIII, eryBI, ermE, eryCI, encodes the following classes of gene products: (1) Three large PK synthase (PKS) enzymes—DEBS 1 (3545 amino acids), DEBS 2 (3567 amino acids), and DEBS 3 (3171 amino acids)—that convert seven equivalents of the propionate-derived monomer methylmalonyl-CoA into the intermediate 6-deoxyerythronolide B (6-DEB); (2) Two P450s that hydroxylate the nascent scaffold; (3) Twelve enzymes that synthesize desosamine and mycarose from glucose and attach them to the nascent scaffold. Without these sugars, erythromycin does not have appreciable antibiotic activity; and (4) An erythromycin resistance gene that modifies the 50S subunit of the ribosome to prevent erythromycin from binding.

Some PKSs have been expressed in heterologous hosts such as E. coli, including erythromycin and the anticancer agent epothilone. The PKS genes have been mutated or replaced with variants from other genes to generate PK derivatives, or to create custom PKSs that synthesize small PK fragments by assembling portions of several PKS genes

Terpenoid Biosynthetic System

Terpenoids are a class of molecules that include the anticancer agent taxol, the antibiotic pleuromutilin, and the carotenoid pigments. While terpenoids are more common among plants than bacteria, carotenoids are produced by a range of bacteria. Lycopene and other carotenoids are generally used in one of two ways: to harvest light (either for energy or photoprotection) or as antioxidants. As with other terpenoids, the first step in the biosynthetic pathway for lycopene is the CrtE-catalyzed polymerization of the C₅ monomer isopentenyl pyrophosphate (IPP) or its Δ² isomer dimethallyl pyrophosphate (DMAPP), in this case to the C₂₀ polymer geranylgeranyl diphosphate (GGDP). CrtB then dimerizes two equivalents of GGDP, resulting in the formation of the linear C₄₀ polymer phytoene. CrtI catalyzes four successive desaturations to yield lycopene. Alternative products such as β-carotene are formed by the action of CrtY, which cyclizes the termini of the linear polyme. In Flavobacterium bacteria, the lycopene biosynthetic system includes the crtE, crtB, crtI, crtY, and crtZ genes. The colored nature of carotenoids has enabled screening of colonies with carotenoid biosynthetic pathways by their color phenotype.

Oligosaccharide Biosynthetic System

Every year, 10,000-20,000 tons of xanthan are produced for use in foods and in industry. Xanthan, an oligosaccharide produced by the plant pathogen Xanthomonas campestris, is composed of a cellulose backbone, on alternating sugars of which a mannose-β-1,4-glucuronate-β-1,2-mannose trisaccharide is appended. A portion of the terminal mannoses have pyruvate linked as a ketal to the 4′- and 6′-hydroxyls, and some of the internal mannoses are acetylated on the 6′-hydroxyl. Owing to the glucuronate units and pyruvoyl substituents, xanthan is an acidic polymer. In Xanthomonas campestris, the biosynthetic system of Xanthan involves the following genes: gumM, gumL, gumK, gumJ, gumI, gumH, gumG, gumF, gumE, gumD, gumC, gumB. Xanthan biosynthesis involves the action of five glycosyltransferases (GumDMHKI), and the growing chain is anchored on undecaprenyl pyrophosphate, similarly to peptidoglycan biosynthesis. Three tailoring enzymes (GumFGL) add the aforementioned pyruvoyl and acetyl substitutents, and GumBCE are required for xanthan export.

Indolocarbazole Biosynthetic System

Indolocarbazoles are natural products formed by the oxidative fusion of primary metabolic monomers. Staurosporine, an indolocarbazole, is a inhibitor of serine/threonine protein kinases that binds in an ATP-competitive manner to these enzymes. In Streptomyces, the biosynthetic system of staurosporine that includes the following genes: staR, staB, staA, staN, staG, staO, staD, staP, staMA, staJ, staK, stal, staE, staMB, staC, encodes three categories of gene products: (1) Four oxidoreductases (two P450s and two flavoenzymes) that catalyze a net 10-electron oxidation to fuse two molecules of tryptophan into the indolocarbazole aglycone; (2) Enzymes to synthesize and attach an unusual hexose to the indolocarbazole scaffold at the indole nitrogens; and (3) A transcriptional activator that regulates the expression of the genes. Other naturally occurring indolocarbazoles differ in the oxidation state of the indolocarbazole scaffold, the derivatization of the indole ring by chlorination, and the sugar substituent appended to the indolocarbazole aglycone.

More than 50 unnatural indolocarbazole derivatives have been made by assembling artificial genes in a non-native host. These molecules harbor chemical modifications that would be difficult to introduce by semisynthetic derivatization of naturally occurring indolocarbazoles or by total synthesis.

Expression Method

A complex biological system (CBS) is a system constituted of multiple genes in an organism that encodes multiple components associated with specific functions or traits, such as nanomachines in an organism, obtaining nutrients and energy from various sources by an organism, metabolic pathways and biosynthesis of natural products, and the like. Genetic engineering of such systems with a large number of genetic components is often difficult, particularly as there is a stoichiometric requirement for balanced expression of the encoded protein components to achieve functions or traits associated with the system. To date, one approach towards engineering CBS involves the complete refactoring of each individual gene, in which all the original native regulatory components have been removed and artificially synthetic regulatory components have been added. The disadvantage of this approach is the increased fragility of refactored systems compared to native systems, and the relative expression levels of multiple proteins encoded by the refactored system are easily affected by various factors, making it difficult to maintain their stoichiometric balance. An alternative approach is to reassemble the system as polycistronic modules, which maintain protein complex stoichiometry. However, large polycistronic operons cannot easily be utilized to express bacterial CBS in eukaryotic cells.

The expression method of the invention involves grouping the components of the complex biological system according to their natural expression levels and constructing fusion expression vectors for each group of genes. Each fusion expression vector constructed expresses a single polyprotein in the cells, which is then cleaved by proteases and releases functional components of the complex biological system. The above method is capable of simplifying the expression procedure of a complex biological system in host cells, reduce the number of vectors that need to be transformed, and maintain the natural stoichiometry between the components. The method of the present invention makes it feasible to exogenously express a functional complex biological system in a host cell, particularly in a eukaryotic cell.

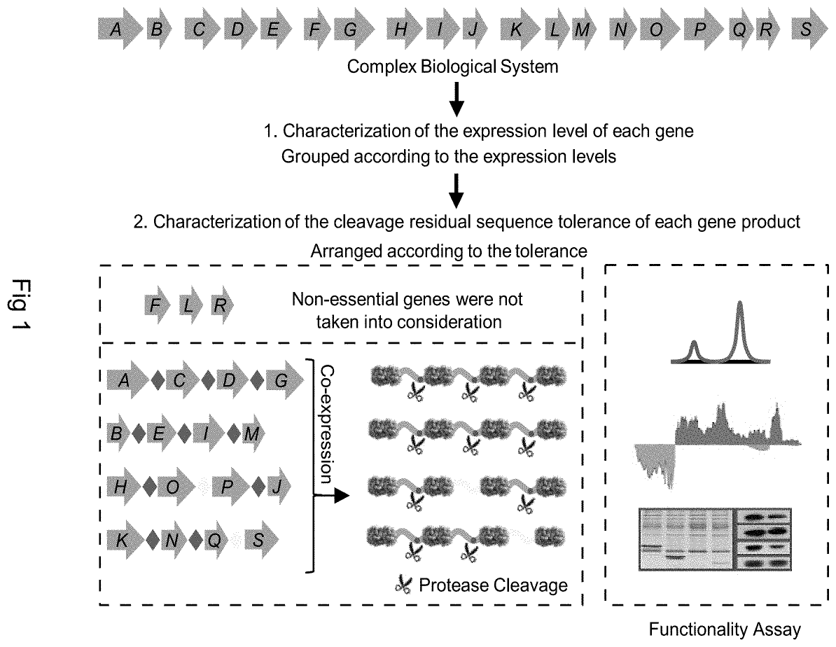

The schematic diagram in FIG. 1 shows exemplary steps for expressing a complex biological system using the method of the present invention.

In one aspect, the invention relates to a method for expressing a complex biological system comprising multiple genes encoding multiple components in a host cell, the method comprising:

a) determining the expression level of each gene in its native operon location;

b) grouping said genes according to the expression level of each gene determined in a), wherein each group comprises genes with similar expression levels;

c) constructing a fusion expression vector for each group of genes according to the grouping in b), wherein the fusion expression vector comprises coding sequences of all genes of its corresponding group, and wherein the coding sequences are directly linked in-frame, linked via a nucleotide sequence encoding a linker, or separated by a nucleotide sequence encoding a cleavage sequence recognized by a protease, thus obtaining a set of fusion expression vectors;

d) introducing the set of fusion expression vectors into a host cell to express a polyprotein from each expression vector;

e) expressing the protease in the host cell to cleave the polyprotein, wherein components encoded by coding sequences directly linked or linked via a nucleotide sequence encoding a linker are expressed as a fusion protein, and wherein components encoded by coding sequences separated by a nucleotide sequence encoding the cleavage sequence are released after protease cleavage.

In some embodiments, the method comprises obtaining a cell or organism that naturally expresses a complex biological system, and determining the expression level of each gene of the complex biological system in the cell or organism.

In some embodiments, the method comprises obtaining a cell or organism that naturally expresses a complex biological system, cloning each gene of the complex biological system into a separate expression vector comprising the native expression control sequence of the gene, transfecting a host cell with the expression vector, and testing the expression level of each gene in the host cell.

In some embodiments, the host cell is a cell line or a model organism. In other embodiments, the host cell is a host cell of interest to be transfected with the complex biological system to express the system therein.

The expression level may be, for example, the level of a transcribed mRNA or the level of a translated protein. The level of mRNA transcribed from a gene can be determined by using, for example, Northern hybridization, RT-PCR, microarray, RNA-seq, and the like. In addition, the level of the translated protein can be determined using Western blotting, or by labeling the protein with a suitable tag (such as His tag, dye, fluorescent substance, isotope, etc.) and quantifying the tag. Various tags and methods for labeling proteins are well known in the art and can be appropriately selected by those skilled in the art as required.

In some embodiments, “having similar expression levels” means that the expression level of any gene is not more than 10 times of that of any of the other genes, preferably the expression level of any gene is not more than 5 times of that of any of the other genes, more preferably the expression level of any gene is not more than 3 times of that of any of the other genes, and even more preferably the expression level of any gene is not more than 2 times of that of any of the other genes.

In embodiments where the coding sequences are linked via a linker, any linker can be used as long as the linker does not affect the activity of the linked protein or polypeptide. A variety of linkers or linker peptide sequences for fusion proteins are known in the art and those skilled in the art can select a suitable linker, such as a flexible linker, according to needs such as the appropriate folding and stability of the protein. In some embodiments, the linker is the sequence (GGGGS)m, wherein m is an integer from 1-10, such as(GGGGS)₅.

In some embodiments of above methods, step c) further comprises testing the activity of the components encoded by genes in each group when expressed as a fusion protein, wherein coding sequences of two or more components that are capable of maintaining the activity of each component when expressed as fusion proteins are directly linked in-frame, or linked via a nucleotide sequence encoding a linker, and other coding sequences are separated by a nucleotide sequence encoding a cleavage sequence recognized by a protease.

In the case where two or more components (such as proteins or polypeptides) are capable of maintaining the function or activity of each component when expressed as a fusion protein, the above components may be expressed and function as a single fusion protein, of which the coding sequences can be linked directly or via a nucleotide sequence encoding a linker. In the cases where two or more components are expressed as a fusion protein and any one of the components is not able to maintain the function or activity of the protein, the coding sequences of the above components are linked by a nucleotide sequence encoding a protease cleavage site. In the presence of a protease (e.g., a protease expressed in a host), the expressed fusion protein is cleaved by the protease and each component is released to perform its respective function.

The expression of complex biological systems and the expression of proteases can be performed simultaneously or sequentially. In some embodiments, the host cell expresses the protease constitutively such that when the fusion protein is expressed, it is immediately cleaved by the protease in the host cell. In other embodiments, the host cell comprises a sequence encoding a protease under the control of an inducible promoter. In this case, each of the components encoded by a complex biological system can be expressed as multiple fusion proteins, and then the expression of the protease can be induced by adding inducers or changing the culture environment. The expressed protease cleaves the fusion proteins to release individual components separated by a protease cleavage sequence.

In some embodiments, being capable of maintaining the activity of each component when expressed as fusion proteins means that when expressed as a fusion protein, the activity of each component is at least 40%, at least 45%, at least 50%, at least 55%, at least 60%, at least 65%, at least 70%, at least 75%, at least 80%, at least 85%, at least 90%, at least 95% or 100% of its activity when expressed as a single protein. In some embodiments, being capable of maintaining the activity of each component when expressed as fusion proteins means that when expressed as a fusion protein, the activity of each component is at least 50%, at least 60%, or at least 70% of its activity when expressed as a single protein. In some embodiments, the activity described above refers to the activity of an enzyme in catalyzing a reaction. In other embodiments, the activity described above refers to other activities of the protein or polypeptide, such as activity and availability as a structural substance of a cell or an organism, activity as a carrier for a transport substance, activity as a cofactor and the like.

In some embodiments of above methods, step c) further comprises a step of arranging coding sequences in a construct, the step comprising testing each component for its tolerance in the presence of a residual sequence at the N-terminal or C-terminal after protease cleavage, wherein for the component with low tolerance in the presence of a residual sequence at the N-terminal, its coding sequence is arranged upstream of the coding sequences of other components; for the component with low tolerance in the presence of a residual sequence at the C-terminal, its coding sequence is arranged downstream of the coding sequences of other components; when there are two or more components with low tolerance in the presence of a residual sequence at the N-terminal in one group, only one of them is retained and its coding sequence is arranged upstream of the coding sequences, and other components with low tolerance in the presence of a residual sequence at the N-terminal are grouped into other groups; when there are two or more components with low tolerance in the presence of a residual sequence at the C-terminal in one group, only one of them is retained and its coding sequence is arranged downstream of the coding sequences, and other components with low tolerance in the presence of a residual sequence at the C-terminal are grouped into other groups.

After a protease recognizes its recognition sequence and cleaves it at the cleavage site, cleavage residual sequences are generally produced at two ends of the cleaved sequences (the N-terminal of one sequence and the C-terminal of the other sequence) with the length ranging from several amino acid residues to tens of amino acid residues. Therefore, the method of the invention further comprises testing whether the presence of a residual sequence at the N-terminal or C-terminal after protease cleavage affects the activity of a component such as a protein or a polypeptide. When the residual sequence at the N-terminal after cleavage affects the activity of the component, the coding sequence of the component is located upstream of the coding sequences of other components in the construct, such that no protease recognition or cleavage sequence is present at the N-terminal of the produced component, and therefore the expression product does not have an N-terminal residual sequence after protease cleavage. Similarly, when the residual sequence at the C-terminal after cleavage affects the activity of the component, the coding sequence of the component is located downstream of the coding sequence of the other components in the construct, such that no protease recognition or cleavage sequence is present at the C-terminal of the produced component, and therefore the expression product does not have an C-terminal residual sequence after protease cleavage. In addition, in the case where there is more than one (for example, two) components in the group that are both sensitive to the N-terminal residue sequence or sensitive to the C-terminal residue sequence, one of the coding sequences is located upstream or downstream in the construct accordingly, and coding sequences of the other components are grouped into other groups. In this way, each component expressed is guaranteed to retain its activity as expressed as a single protein.

In some embodiments, the component with low tolerance in the presence of a residual sequence at the N-terminal or C-terminal is defined as that the activity of the component is reduced by at least 10%, at least 20%, at least 30%, at least 40%, at least 50%, at least 60%, at least 70%, at least 80%, or at least 90% in the presence of a residual sequence at its N-terminal or C-terminal. In some embodiments, the component with low tolerance in the presence of a residual sequence at the N-terminal or C-terminal is defined as: (activity in the presence of a residual sequence/activity in the absence of a residual sequence %)ⁿ is less than 30%, less than 40%, less than 50%, less than 60%, less than 70%, less than 80%, or less than 90%, wherein n is the number of genes of said complex biological system. In some embodiments, the activity described above refers to the activity of an enzyme in catalyzing a reaction. In other embodiments, the activity described above refers to other activities of the protein or polypeptide, such as activity and availability as a structural substance of a cell or an organism, activity as a carrier for a transport substance, activity as a cofactor and the like.

In any embodiment of above methods, genes originally with different expression levels achieve similar expression levels by adjusting the copy number of coding sequences and are grouped into one group. For example, in the case where the expression level of a first gene is about 2 times that of a second gene, the copy number of the coding sequence of the second gene may be adjusted to 2 and the above first and second genes are grouped into the same group. The above expression level may refer to the expression level of a gene in its native operon location. The step of increasing the copy number of a gene is particularly applicable when the natural expression level of one component is about an integer multiple of another component, such as about 2 or 3 times.

In any embodiment of above methods, for one or more fusion expression vectors, for example, each of the fusion expression vectors may use a native expression control sequence of one of genes in its corresponding group or another expression control sequence having a similar expression level therewith. Said another expression control sequence may be an expression control sequence from other genes, or a synthetic expression control sequence.

In the above method, any suitable protease can be used. In some embodiments, the protease is selected from the group consisting of thrombin, Factor Xa, enterokinase, Tobacco Etch Virus (TEV) protease, PreScission protease and HRV 3C protease. In some embodiments, the protease is TEV protease.

Thrombin, also known as cellulase, is a serine protease that is encoded by F2 gene in human. During coagulation, prothrombin (coagulation factor II) is proteolytically cleaved to form thrombin, which functions as a serine protease and converts soluble fibrinogen into insoluble fibrin chains. The recognition sequence of thrombin is LVPRG↓S, wherein ↓ represents the cleavage site.

Factor Xa, also known as coagulation factor Xa, is a glycosylated serine protease and a key enzyme in the coagulation process. During coagulation, factor X is activated by hydrolysis to form factor Xa. Factor Xa and Va form a prothrombin complex, which can convert prothrombin to thrombin. The recognition sequence of factor Xa is IE/DG↓R, wherein ↓ represents the cleavage site.

Enteropeptidase, also known as enterokinase, is an enzyme that is produced by the duodenal cells and is involved in digestion in humans and other animals. It is a serine protease that converts trypsinogen (a kind of zymogen) to its active form trypsin, resulting in subsequent activation of pancreatic digestive enzymes. Its recognition sequence is DDDDK↓, wherein ↓ represents the cleavage site.

TEV protease (Tobacco etch virus nuclear inclusion-a endopeptidase) is a highly sequence-specific cysteine protease derived from tobacco etch virus and is commonly used for controlled cleavage of fusion proteins in vivo and in vitro. Its recognition sequence is ENLYFQ↓S/G, wherein ↓ represents the cleavage site.

PreScission protease is a fusion protein of glutathione S-transferase (GST) and human rhinovirus (HRV) type 14 3C protease. This protease specifically recognizes and cleaves the sequence LEVLFQ↓GP, wherein ↓ represents the cleavage site. Its substrate recognition and cleavage depends not only on the primary structure of the fusion protein, but also on the secondary and tertiary structure of the fusion protein.

HRV 3C protease is a recombinant 3C protease encoded by human rhinovirus 14 recombinantly obtained from E. coli. Its recognition sequence is LEVLFQ↓GP, wherein ↓ represents the cleavage site.

In some embodiments of above methods, the host cell is a prokaryotic cell or a eukaryotic cell. For example, the prokaryotic cell may be selected from Pseudomonas fluorescens, Bacillus subtilis, Pseudomonas protegens, Pseudomonas putida, Pseudomonas veronii, Pseudomonas taetrolens, Pseudomonas balearica, Pseudomonas stutzeri, Pseudomonas aeruginosa, Pseudomonas syringae, Bacillus amyloliquefaciens, Burkholderia phytofirmans, Gluconacetobacter diazotrophicus, Herbaspirillum seropedicae, Bacillus cereus. For example, the eukaryotic cell may be, for example, selected from the cell of following species: Oryza sativa, Triticum aestivum, Zea mays, Sorghum bicolor, Setaria italica, Solanum tuberosum, Ipomoea batatas, Arachis hypogaea, Brassica napus, Malva farviflora, Sesamum indicum, Olea europaea, Elaeis guineensis, Saccharum officinarum, Beta vulgaris, Gossypium spp.

The method of the invention can be used to express any complex biological system in a host cell. In some embodiments, the method of the invention may be used to express the complex biological system selected from the group consisting of alkane degradation pathway, nitrogen fixation system, polychlorinated biphenyl degradation system, bioplastic biosynthetic system (poly(3-hydroxybutryrate) biosynthetic system), nonribosomal peptide biosynthetic system, polyketide biosynthetic system, terpenoid biosynthetic system, oligosaccharide biosynthetic system, indolocarbazole biosynthetic system.

The complex biological system described above is not limited to a specific species source, and may be derived from different categories of cells or organisms. A variety of cells and organisms with such complex biological systems are known in the art, for example as described in the “Complex Biological System” section.

In some embodiments, the complex biological system is a nitrogen fixation system. In some embodiments, the nitrogen-fixing cell comprises the following genes: nifH, nifD, nifK, nifY, nifE, nifN, nifX, nifB, nifU, nifU, nifS, nifV, nifM, nifJ, nifF and optionally nifT, nifX, nifQ, nifW, nifZ. In some embodiments, the nitrogen fixation system is from Klebsiella oxytoca.

The nitrogen fixation system of Klebsiella oxytoca is composed of 17-20 nif genes, which are mainly: J, H, D, K, T, Y, E, N, X, U, S, V, W, Z, M, F, L, A, B and Q, constituting the following seven operons:

NifJ operon: comprising nifJ gene;

NifHDKY operon: comprising nifH, nifD, nifK and nifY genes;

NifENX operon: comprising nifE, nifN and nifX genes;

NifUSVM operon: comprising nifU, nifS, nifC and nifM genes;

NifF operon: comprising nifF gene;

NifLA operon: comprising nifL, nifA genes;

NifBQ operon: comprising nifB, nifQ genes.

Among all nitrogen-fixing microorganisms, the entire nitrogen fixation system is relatively conservative, and nitrogen-fixing genes between different organisms also have high homology. For example, the nif genes in the nitrogen-fixing gene system of rhizobia are homologous to those of Klebsiella oxytoca. Therefore, the present invention is not limited to expression of nitrogen fixation systems from Klebsiella oxytoca, and includes nitrogen fixation systems from other species.

In order to minimize the number of genes in the nitrogen fixation system and simplify the arrangement of the polyproteins encoded by the genes, the nifT, nifX, nifW, and nifZ genes may be omitted because these genes have been shown to be unnecessary for biological nitrogen fixation systems in E. coli. In addition, it is known that the activity of the nitrogen fixation system can be restored in the absence of nifQ gene by exogenously supplying molybdenum. Therefore, nifQ gene can also be omitted.

In any embodiment of above methods, according to factors such as the number of genes in the complex biological systems, the expression level and the tolerance to terminal residual sequences after protease cleavage of each gene, the activity of each component when expressed as a fusion protein, and the type of the host cell to be transfected, the genes of the complex biological systems may be grouped into several groups. The number of groups is not limited, and in some embodiments, in particular if the complex biological system is a nitrogen fixation system, genes may be grouped into three to seven groups, for example, three groups, four groups, five groups, six groups or seven groups. In some embodiments, the genes can be grouped into four groups, five groups or six groups.

By using the nitrogen fixation system as an example of a complex biological system, the invention investigates grouping genes of the nitrogen fixation system and expressing them in a host cell by the method of present invention.

In some embodiments of above methods of expressing a nitrogen fixation system, the following genes are grouped into one group: nifH, nifD, nifK. In some embodiments, nifH, nifD, nifK genes are grouped into one group and their corresponding fusion expression vector has the following manner of arrangement and connection from upstream to downstream: nifH-cleav-nifD-cleav-nifK, wherein cleav is a nucleotide sequence encoding a cleavage sequence recognized by a protease.

In some embodiments, the following genes are grouped into one group: nifE, nifN, nifB. In some embodiments, nifE, nifN, nifB genes are grouped into one group and their corresponding fusion expression vector has the following manner of arrangement and connection from upstream to downstream: nifE-cleav-nifN-linker-nifB, wherein cleav is a nucleotide sequence encoding a cleavage sequence recognized by a protease, and linker is a nucleotide sequence encoding a linker. In a preferable embodiment, the linker is (GGGGS)m, wherein m is an integer from 1-10. For example, the linker may be (GGGGS)₅.

In some embodiments, the following genes are grouped into one group: nifF, nifM, nifY. In some embodiments, nifF, nifM, nifY genes are grouped into one group and their corresponding fusion expression vector has the following manner of arrangement and connection from upstream to downstream: nifF-cleav-nifM-cleav-nifY, wherein cleav is a nucleotide sequence encoding a cleavage sequence recognized by a protease.

In some embodiments, the following genes are grouped into one group: nifJ, nifV and optionally nifW, nifZ. In some embodiments, the fusion expression vector corresponding to the above gene grouping has the following structure from upstream to downstream: nifJ-cleav-nifV-cleav-nifW, nifJ-cleav-nifV-cleav-nifZ, or nifJ-cleav-nifV-cleav-nifW-cleav-nifZ, wherein cleav is a nucleotide sequence encoding a cleavage sequence recognized by a protease.

In any embodiment of above methods, nifU and nifS genes are grouped into one group, or nifU and nifS are expressed as independent genes. In an embodiment in which nifU and nifS genes are grouped into one group, the fusion expression vector comprising the coding sequences of nifU and nifS genes has the following manner of arrangement and connection from upstream to downstream: nifU-cleav-nifS, wherein cleav is a nucleotide sequence encoding a cleavage sequence recognized by a protease.

In a further embodiment, the coding sequences of nifH, nifD, nifK, nifY, nifE, nifN, nifB, nifU, nifS, nifV, nifM, nifJ, nifF and optionally nifW, nifZ genes of a nitrogen fixation system are cloned into five fusion expression vectors in the following manner of arrangement and connection:

a) nifH-cleav-nifD-cleav-nifK;

b) nifE-cleav-nifN-linker-nifB;

c) nifU-cleav-nifS;

d) nifJ-cleav-nifV-cleav-nifW, or nifJ-cleav-nifV-cleav-nifZ; and

e) nifF-cleav-nifM-cleav-nifY,

wherein cleav is a nucleotide sequence encoding a cleavage sequence recognized by a protease, and linker is a nucleotide sequence encoding a linker.

In other embodiments, the coding sequences of nifH, nifD, nifK, nifY, nifE, nifN, nifB, nifU, nifS, nifV, nifM, nifJ, nifF and nifW genes of a nitrogen fixation system are cloned into six fusion expression vectors in the following manner of arrangement and connection:

a) nifH-cleav-nifD-cleav-nifK;

b) nifE-cleav-nifN-linker-nifB;

c) nifU;

d) nifS;

e) nifJ-cleav-nifV-cleav-nifW; and

f) nifF-cleav-nifM-cleav-nifY,

wherein cleav is a nucleotide sequence encoding a cleavage sequence recognized by a protease, and linker is a nucleotide sequence encoding a linker.

In some embodiments, the coding sequences of nifH, nifD, nifK, nifY, nifE, nifN, nifB, nifU, nifS, nifV, nifM, nifJ and nifF genes of a nitrogen fixation system are cloned into 5 fusion expression vectors in the following manner of arrangement and connection:

a) nifH-cleav-nifD-cleav-nifK;

b) nifE-cleav-nifN-linker-nifB;

c) nifU-cleav-nifS-cleav-nifV;

d) nifJ; and

e) nifF-cleav-nifM-cleav-nifY,

wherein cleav is a nucleotide sequence encoding a cleavage sequence recognized by a protease, and linker is a nucleotide sequence encoding a linker.

In some embodiments, the coding sequences of nifH, nifD, nifK, nifY, nifE, nifN, nifB, nifU, nifS, nifV, nifM, nifJ and nifF genes of a nitrogen fixation system are cloned into five fusion expression vectors in the following manner of arrangement and connection:

a) nifH-cleav-nifD-cleav-nifK;

b) nifE-cleav-nifN-linker-nifB;

c) nifU-cleav-nifS;

d) nifJ-cleav-nifV; and

e) nifF-cleav-nifM-cleav-nifY,

wherein cleav is a nucleotide sequence encoding a cleavage sequence recognized by a protease, and linker is a nucleotide sequence encoding a linker.

In some embodiments, the coding sequences of nifH, nifD, nifK, nifY, nifE, nifN, nifB, nifU, nifS, nifV, nifM, nifJ, nifF, nifW and nifZ genes of a nitrogen fixation system are cloned into five fusion expression vectors in the following manner of arrangement and connection:

a) nifH-cleav-nifD-cleav-nifK;

b) nifE-cleav-nifN-linker-nifB;

c) nifU-cleav-nifS;

d) nifJ-cleav-nifV-cleav-nifW-cleav-nifZ; and

e) nifF-cleav-nifM-cleav-nifY,

wherein cleav is a nucleotide sequence encoding a cleavage sequence recognized by a protease, and linker is a nucleotide sequence encoding a linker.

In some embodiments, the coding sequences of nifH, nifD, nifK, nifY, nifE, nifN, nifB, nifU, nifS, nifV, nifM, nifJ, nifF and nifW genes of a nitrogen fixation system are cloned into six fusion expression vectors in the following manner of arrangement and connection:

a) nifH-cleav-nifD-cleav-nifK;

b) nifE-cleav-nifN-linker-nifB;

c) nifU-cleav-nifS;

d) nifJ;

e) nifF; and

f) nifV-cleav-nifW-cleav-nifM-cleav-nifY,

wherein cleav is a nucleotide sequence encoding a cleavage sequence recognized by a protease, and linker is a nucleotide sequence encoding a linker.

In some embodiments, the coding sequences of nifH, nifD, nifK, nifY, nifE, nifN, nifB, nifU, nifS, nifV, nifM, nifJ, nifF and nifW genes of a nitrogen fixation system are cloned into seven fusion expression vectors in the following manner of arrangement and connection:

a) nifH-cleav-nifD-cleav-nifK;

b) nifE-cleav-nifN-linker-nifB;

c) nifU;

d) nifS;

e) nifJ;

f) nifF; and

g) nifV-cleav-nifW-cleav-nifM-cleav-nifY,

wherein cleav is a nucleotide sequence encoding a cleavage sequence recognized by a protease, and linker is a nucleotide sequence encoding a linker.

Vector

In another aspect, the invention relates to vectors, such as expression vectors, which can be used to express complex biological systems comprising multiple genes in a host cell.

In some embodiments, the invention relates to a vector comprising coding sequences of two or more genes of a complex biological system, said complex biological system comprises multiple genes encoding multiple components, said two or more genes have similar expression levels in their native operon locations, wherein the coding sequences of the two or more genes are directly linked in-frame, linked via a nucleotide sequence encoding a linker, or separated by a nucleotide sequence encoding a cleavage sequence recognized by a protease.

The complex biological system can be any complex biological system, such as those described in the “Complex Biological System” section.

The vector may be any vector, and examples include, for example, a vector derived from a bacterial plasmid, a viral vector, a vector derived from a yeast plasmid, a vector derived from a phage, a cosmid, a phagemid, and the like.

In some embodiments, the vector is an expression vector, such as a fusion expression vector. In other embodiments, the vector is a cloning vector.

In addition to the coding sequence of a gene, a vector such as an expression vector may comprise a promoter and expression control sequences such as transcription and termination signals. The vector may also include one or more restriction sites to allow insertion of the coding sequences at these sites. The coding sequence can be expressed by inserting the coding sequence or a nucleic acid construct comprising the coding sequence into an expression vector. When preparing the expression vector, the coding sequence is located in the vector such that the coding sequence is operatively linked to the expression control sequence. A recombinant expression vector can be any vector (e.g., a plasmid or virus) that can be conveniently subjected to recombinant DNA procedures and can facilitate expression of a polynucleotide. The selection of the vector will typically depend on the compatibility of the vector with the host cell into which the vector is to be introduced. The vector can be a linear or closed circular plasmid.

The vector may be an autonomous replication vector, that is, a vector that exists as an extrachromosomal entity, and its replication is independent of chromosomal replication, such as a plasmid, extrachromosomal element, minichromosome, or artificial chromosome. The vector may contain any element for ensuring self-replication. Alternatively, the vector may be an integration vector that, when introduced into a host cell, is integrated into the genome and replicated with one or more chromosomes. In addition, a single vector or two or more vectors may be used.

For autonomous replication, the vector may further comprise an origin of replication that enables the vector to autonomously replicate in the host cell. The origin of replication can be any plasmid replicon that functions in a cell to initiate autonomous replication. The term “origin of replication” or “plasmid replicon” means a polynucleotide that enables a plasmid or vector to replicate in vivo.

Examples of origins of replication for bacteria are those of plasmids pBR322, pUC19, pACYC177, and pACYC184 that allow replication in E. coli, and plasmids pUB110, pE194, pTA1060, and pAMI31 that allow replication in Bacillus.

Examples of origins of replication used in yeast host cells are 2 μm origin of replication, ARS1, ARS4, a combination of ARS1 and CEN3, and a combination of ARS4 and CEN6.

For a vector integrated into the host cell genome, the vector can be integrated into the genome by homologous recombination. In this case, the vector may contain a polynucleotide for directing integration into the genome of the host cell at one or more precise locations on one or more chromosomes by homologous recombination. To increase the possibility of integration at precise locations, the integration element should contain a sufficient number of nucleotides that have high sequence identity with the corresponding target sequence to enhance the possibility of homologous recombination. These integration elements can be any sequence that is homologous to a target sequence in the host cell genome. In addition, these integration elements may be non-coding polynucleotides or coding polynucleotides. In another aspect, the vector can be integrated into the genome of the host cell by non-homologous recombination.

The vector may contain one or more selectable markers that allow easy selection of transformed cells, transfected cells, transduced cells, and the like. A selectable marker is a gene of which product provides biocide resistance or virus resistance, resistance to heavy metals, prototrophy to auxotrophs, and the like.

Examples of bacterial selectable markers include markers for dal gene of Bacillus licheniformis or Bacillus subtilis, or those conferring antibiotic resistance (such as ampicillin, chloramphenicol, kanamycin, neomycin, spectinomycin, or tetracycline resistance). Suitable markers for use in yeast host cells include but are not limited to ADE2, HIS3, LEU2, LYS2, MET3, TRP1, and URA3. Selectable markers for use in a filamentous fungal host cell include, but are not limited to, adeA (phosphoribosylaminoimidazole-succinocarboxamide synthase), adeB (phosphoribosyl-aminoimidazole synthase), amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), etc.

In some embodiments, “two or more genes have similar expression levels in their native operon location” means that the two or more genes have similar expression levels in a cell or organism that naturally expresses the complex biological system, such as the level of transcribed mRNA or the level of translated protein.

In other embodiments, “two or more genes have similar expression levels in their native operon location” means that the two or more genes have similar expression levels when cloned into an expression vector comprising their corresponding native expression control sequences and expressed in a host cell. In some embodiments, the host cell is a cell line or a model organism. In other embodiments, the host cell is a host cell of interest to be transfected with the complex biological system to express the system therein.

In some embodiments, “having similar expression levels” means that the expression level of any gene is not more than 10 times of that of any of other genes, preferably the expression level of any gene is not more than 5 times of that of any of other genes, more preferably the expression level of any gene is not more than 3 times of that of any of other genes, and even more preferably the expression level of any gene is not more than 2 times of that of any of other genes.

In embodiments where the coding sequences are linked via a linker, any linker can be used as long as the linker does not affect the activity of the linked proteins or polypeptides. In some embodiments, the linker is (GGGGS)m, wherein m is an integer from 1-10. In some embodiments, the linker is (GGGGS)₅.

In some embodiments, in said vector, coding sequences of two or more components that are capable of maintaining the activity of each component when expressed as fusion proteins are directly linked in-frame, or linked via a nucleotide sequence encoding a linker, and other coding sequences are separated by a nucleotide sequence encoding a cleavage sequence recognized by a protease. In some embodiments, being capable of maintaining the activity of each component when expressed as fusion proteins means that when expressed as a fusion protein, the activity of each component is at least 30%, at least 35%, at least 40%, at least 45%, at least 50%, at least 55%, at least 60%, at least 65%, at least 70%, at least 75%, at least 80%, at least 85%, at least 90%, at least 95% or 100% of its activity when expressed as a single protein. In some embodiments, being capable of maintaining the activity of each component when expressed as a fusion protein means that when expressed as a fusion protein, the activity of each component is at least 50%, at least 60%, or at least 70% of its activity when expressed as a single protein. In some embodiments, the activity is an enzymatic activity. In other embodiments, the activity described above refers to other activities of the protein or polypeptide, such as activity and availability as a structural substance of a cell or an organism, activity as a carrier for a transport substance, and activity as a cofactor.

In some embodiments of above vectors, the coding sequence of a component with low tolerance in the presence of a residual sequence at the N-terminal after protease cleavage is arranged upstream of the coding sequences of other components; the coding sequence of a component with low tolerance in the presence of a residual sequence at the C-terminal is arranged downstream of the coding sequences of other components. In some embodiments, the component with low tolerance in the presence of a residual sequence at the N-terminal or C-terminal is defined as that the activity of the component is reduced by at least 10%, at least 20%, at least 30%, at least 40%, at least 50%, at least 60%, at least 70%, at least 80%, or at least 90% in the presence of a residual sequence at its N-terminal or C-terminal. In some embodiments, the component with low tolerance in the presence of a residual sequence at the N-terminal or C-terminal is defined as: (activity in the presence of a residual sequence/activity in the absence of a residual sequence %)ⁿ is less than 30%, less than 40%, less than 50%, less than 60%, less than 70%, less than 80%, or less than 90%, wherein n is the number of genes of said complex biological system.

In some embodiments, the activity is an enzymatic activity. In other embodiments, the activity described above refers to other activities of the protein or polypeptide, such as activity and availability as a structural substance of a cell or an organism, activity as a carrier for a transport substance, and activity as a cofactor.

In any embodiment of above vectors, the vector includes different copy numbers of coding sequences of two or more genes, so that genes originally with different expression levels achieve similar expression levels. For example, in the case where the expression level of a first gene is about 2 times that of a second gene, the copy number of the coding sequence of the second gene may be adjusted to 2 and the above first and second genes are grouped into the same group. The above expression level refers to the expression level of a gene in its native operon location. The above embodiments are particularly applicable where the expression level of one gene is about an integer multiple of another gene, for example, the expression level of one gene is about 2 or about 3 times that of another gene.

In any embodiment of above vectors, especially in the case where the vector is an expression vector, the vector may have a native expression control sequence of one of the two or more genes or another expression control sequence having a similar expression level therewith. Said another expression control sequence may be an expression control sequence from other genes, or an artificially synthetic expression control sequence.

In any embodiment of above vectors, the protease is selected from the group consisting of thrombin, Factor Xa, enterokinase, Tobacco Etch Virus (TEV) protease, PreScission and HRV 3C protease. In some embodiments, the protease is TEV protease.

In some embodiments, the vector is an expression vector for expression in a host cell, and the host cell may be a prokaryotic cell or a eukaryotic cell. Examples of the prokaryotic cells include, for example, Pseudomonas fluorescens, Bacillus subtilis, Pseudomonas protegens, Pseudomonas putida, Pseudomonas veronii, Pseudomonas taetrolens, Pseudomonas balearica, Pseudomonas stutzeri, Pseudomonas aeruginosa, Pseudomonas syringae, Bacillus amyloliquefaciens, Burkholderia phytofirmans, Gluconacetobacter diazotrophicus, Herbaspirillum seropedicae, Bacillus cereus. Examples of the eukaryotic cells include, for example, cells selected from the following species: Oryza sativa, Triticum aestivum, Zea mays, Sorghum bicolor, Setaria italica, Solanum tuberosum, Ipomoea batatas, Arachis hypogaea, Brassica napus, Malva farviflora, Sesamum indicum, Olea europaea, Elaeis guineensis, Saccharum officinarum, Beta vulgaris, Gossypium spp.

In any embodiment of above vectors, the complex biological system is selected from alkane degradation pathway, nitrogen fixation system, polychlorinated biphenyl degradation system, bioplastic biosynthetic system (poly(3-hydroxybutryrate) biosynthetic system), nonribosomal peptide biosynthetic system, polyketide biosynthetic system, terpenoid biosynthetic system, oligosaccharide biosynthetic system, indolocarbazole biosynthetic system.

The complex biological system described above is not limited to a specific species source, and may be derived from different categories of cells or organisms. A variety of cells and organisms with such complex biological systems are known in the art, for example as described in the “Complex Biological System” section.

In some embodiments, the complex biological system is a nitrogen fixation system.

In some embodiments, the nitrogen fixation system comprises the following genes: nifH, nifD, nifK, nifY, nifE, nifN, nifX, nifB, nifU, nifU, nifS, nifV, nifM, nifJ, nifF and optionally nifT, nifX, nifQ, nifW, nifZ.

In some embodiments, the nitrogen fixation system is from Klebsiella oxytoca.

In some embodiments, the vector comprises coding sequences of the following genes: nifH, nifD, nifK. Preferably, the vector has the following manner of arrangement and connection from upstream to downstream: nifH-cleav-nifD-cleav-nifK, wherein cleav is a nucleotide sequence encoding a cleavage sequence recognized by a protease.

In some embodiments, the vector comprises coding sequences of the following genes: nifE, nifN, nifB. Preferably, the vector has the following manner of arrangement and connection from upstream to downstream: nifE-cleav-nifN-linker-nifB, wherein cleav is a nucleotide sequence encoding a cleavage sequence recognized by a protease, and linker is a nucleotide sequence encoding a linker. In some embodiments, the linker is (GGGGS)m, wherein m is an integer from 1-10. For example, the linker may be (GGGGS)₅.

In some embodiments, the vector comprises coding sequences of the following genes: nifF, nifM, nifY. Preferably, the vector has the following manner of arrangement and connection from upstream to downstream: nifF-cleav-nifM-cleav-nifY, wherein cleav is a nucleotide sequence encoding a cleavage sequence recognized by a protease.

In some embodiments, the vector comprises coding sequences of the following genes: nifJ, nifV and optionally nifW, nifZ. Preferably, the vector has the following manner of arrangement and connection from upstream to downstream: nifJ-cleav-nifV-cleav-nifW, nifJ-cleav-nifV-cleav-nifZ, or nifJ-cleav-nifV-cleav-nifW-cleav-nifZ, wherein cleav is a nucleotide sequence encoding a cleavage sequence recognized by a protease.

In some embodiments, the vector comprises coding sequences of the following genes: nifU, nifS. Preferably, the vector has the following manner of arrangement and connection from upstream to downstream: nifU-cleav-nifS, wherein cleav is a nucleotide sequence encoding a cleavage sequence recognized by a protease.

Vector Composition

In yet another aspect, the invention relates to a vector composition comprising multiple vectors each comprising a coding sequence of one or more genes of a complex biological system, said complex biological system comprising multiple genes encoding multiple components, wherein in a vector comprising coding sequences of two or more genes, said two or more genes have similar expression levels in their native operon locations, wherein the coding sequences of the two or more genes are directly linked in-frame, linked via a nucleotide sequence encoding a linker, or separated by a nucleotide sequence encoding a cleavage sequence recognized by a protease.

In some embodiments, the coding sequence of each gene of the complex biological system is present in one of the vectors of the vector composition.

In some embodiments, the multiple vectors of the vector composition collectively comprise coding sequences of all genes of the complex biological system.

The complex biological system can be any complex biological system, such as those described in the “Complex Biological System” section.

The vector may be any vector, and examples thereof include, for example, a vector derived from a bacterial plasmid, a viral vector, a vector derived from a yeast plasmid, a vector derived from a phage, a cosmid, a phagemid, and the like. In some embodiments, the multiple vectors in the vector composition are vectors of the same type, such as a plasmid. In other embodiments, the vector composition has different types of vectors, such as plasmid vectors and viral vectors. In some embodiments, the multiple vectors in the vector composition have the same backbone structure. In other embodiments, multiple vectors in the vector composition have different backbone structures.

In some embodiments, “two or more genes have similar expression levels in their native operon location” means that the two or more genes have similar expression levels in a cell or organism that naturally expresses the complex biological system, such as the level of transcribed mRNA or the level of translated protein.

In other embodiments, “two or more genes have similar expression levels in their native operon location” means that the two or more genes have similar expression levels when cloned into an expression vector comprising their corresponding native expression control sequences and expressed in a host cell. In some embodiments, the host cell is a cell line or a model organism. In other embodiments, the host cell is a host cell of interest to be transfected with the complex biological system to express the system therein.

In some embodiments, “having similar expression levels” means that the expression level of any gene is not more than 10 times of that of any of other genes, preferably the expression level of any gene is not more than 5 times of that of any of other genes, more preferably the expression level of any gene is not more than 3 times of that of any of other genes, and even more preferably the expression level of any gene is not more than 2 times of that of any of other genes.

In embodiments where the coding sequences are linked via a linker, any linker can be used as long as the linker does not affect the activity of the linked proteins or polypeptides. In some embodiments, the linker is (GGGGS)m, wherein m is an integer from 1-10. In some embodiments, the linker is (GGGGS)₅.

In some embodiments of the vector composition, in a vector comprising coding sequences of two or more genes, coding sequences of genes of two or more components that are capable of maintaining the activity of each component when expressed as fusion proteins are directly linked in-frame, or linked via a nucleotide sequence encoding a linker, and other components are separated by a nucleotide sequence encoding a cleavage sequence recognized by a protease.

In some embodiments, being capable of maintaining the activity of each component when expressed as fusion proteins means that when expressed as a fusion protein, the activity of each component is at least 40%, at least 45%, at least 50%, at least 55%, at least 60%, at least 65%, at least 70%, at least 75%, at least 80%, at least 85%, at least 90%, at least 95% or 100% of its activity when expressed as a single protein. In some embodiments, being capable of maintaining the activity of each component when expressed as a fusion protein means that when expressed as a fusion protein, the activity of each component is at least 50%, at least 60%, or at least 70% of its activity when expressed as a single protein. In some embodiments, the activity is an enzymatic activity. In other embodiments, the activity described above refers to other activities of the protein or polypeptide, such as activity and availability as a structural substance of a cell or an organism, activity as a carrier for a transport substance, and activity as a cofactor.

In some embodiments of the vector composition, in a vector comprising coding sequences of two or more genes, the coding sequence of a component with low tolerance in the presence of a residual sequence at the N-terminal after protease cleavage is arranged upstream of the coding sequences of other components; the coding sequence of a component with low tolerance in the presence of a residual sequence at the C-terminal is arranged downstream of the coding sequences of other components. In some embodiments, the component with low tolerance in the presence of a residual sequence at the N-terminal or C-terminal is defined as that the activity of the component is reduced by at least 40%, at least 45%, at least 50%, at least 55%, at least 60%, at least 65%, at least 70%, at least 75%, at least 80%, at least 85%, at least 90%, at least 95% or 100% in the presence of a residual sequence at its N-terminal or C-terminal. In some embodiments, the component with low tolerance in the presence of a residual sequence at the N-terminal or C-terminal is defined as that the activity of the component is reduced by at least 50%, at least 60%, or at least 70% in the presence of residual sequences at its N-terminal or C-terminal.

In some embodiments, the activity is an enzymatic activity. In other embodiments, the activity described above refers to other activities of the protein or polypeptide, such as activity and availability as a structural substance of a cell or an organism, activity as a carrier for a transport substance, and activity as a cofactor.

In some embodiments of the vector composition, in a vector comprising coding sequences of two or more genes, genes originally with different expression levels achieve similar expression levels by comprising different copy numbers of coding sequences. The above embodiments are particularly applicable where the expression level of one gene is about an integer multiple of another gene, for example, the expression level of one gene is about 2 or about 3 times that of another gene.

In some embodiments, one or more vectors in the vector composition, for example, each of the vectors has an expression control sequence of one of the coding sequences of the one or more components comprised therein or another expression control sequence having a similar expression level therewith. Said another expression control sequence may be an expression control sequence from other genes, or an artificially synthetic expression control sequence.

In any embodiment of the vector composition, the protease may be selected from the group consisting of thrombin, Factor Xa, enterokinase, Tobacco Etch Virus (TEV) protease, PreScission and HRV 3C protease. In some embodiments, the protease is TEV protease.

In some embodiments, one or more vectors in the vector composition, for example, each of the vectors is a fusion expression vector for expression in a host cell. In some embodiments, the host cell is a prokaryotic cell or a eukaryotic cell. Examples of the prokaryotic cells include, for example, Pseudomonas fluorescens, Bacillus subtilis, Pseudomonas protegens, Pseudomonas putida, Pseudomonas veronii, Pseudomonas taetrolens, Pseudomonas balearica, Pseudomonas stutzeri, Pseudomonas aeruginosa, Pseudomonas syringae, Bacillus amyloliquefaciens, Burkholderia phytofirmans, Gluconacetobacter diazotrophicus, Herbaspirillum seropedicae, Bacillus cereus, etc. Examples of the eukaryotic cells include, for example, cells selected from the following species: Oryza sativa, Triticum aestivum, Zea mays, Sorghum bicolor, Setaria italica, Solanum tuberosum, Ipomoea batatas, Arachis hypogaea, Brassica napus, Malva farviflora, Sesamum indicum, Olea europaea, Elaeis guineensis, Saccharum officinarum, Beta vulgaris, Gossypium spp.

In any embodiment of the vector compositions, the complex biological system may be selected from: alkane degradation pathway, nitrogen fixation system, polychlorinated biphenyl degradation system, bioplastic biosynthetic system (poly(3-hydroxybutryrate) biosynthetic system), nonribosomal peptide biosynthetic system, polyketide biosynthetic system, terpenoid biosynthetic system, oligosaccharide biosynthetic system, indolocarbazole biosynthetic system.

The complex biological system described above is not limited to a specific species source, and may be derived from different categories of cells or organisms. A variety of cells and organisms with such complex biological systems are known in the art, for example as described in the “Complex Biological System” section.

In some embodiments, the complex biological system is a nitrogen fixation system.

In some embodiments, the nitrogen fixation system comprises the following genes: nifH, nifD, nifK, nifY, nifE, nifN, nifX, nifB, nifU, nifU, nifS, nifV, nifM, nifJ, nifF and optionally nifT, nifX, nifQ, nifW, nifZ.

In some embodiments, the nitrogen fixation system is from Klebsiella oxytoca.

In any embodiment of the vector compositions, the vector composition comprises three to seven vectors, for example three, four, five, six or seven vectors. In some embodiments, the vector composition comprises four, five or six vectors.

In some embodiments, the vector composition comprises a vector comprising coding sequences of the following genes: nifH, nifD, nifK. Preferably, the vector has the following manner of arrangement and connection from upstream to downstream: nifH-cleav-nifD-cleav-nifK, wherein cleav is a nucleotide sequence encoding a cleavage sequence recognized by a protease.

In some embodiments, the vector composition comprises a vector comprising coding sequences of the following genes: nifE, nifN, nifB. Preferably, the vector has the following manner of arrangement and connection from upstream to downstream: nifE-cleav-nifN-linker-nifB, wherein cleav is a nucleotide sequence encoding a cleavage sequence recognized by a protease, and linker is a nucleotide sequence encoding a linker. In some embodiments, the linker is (GGGGS)m, wherein m is an integer from 1-10, such as (GGGGS)₅.

In some embodiments, the vector composition comprises a vector comprising coding sequences of the following genes: nifF, nifM, nifY. Preferably, the vector has the following manner of arrangement and connection from upstream to downstream: nifF-cleav-nifM-cleav-nifY, wherein cleav is a nucleotide sequence encoding a cleavage sequence recognized by a protease.

In some embodiments, the vector composition comprises a vector comprising coding sequences of the following genes: nifJ, nifV and optionally nifW, nifZ. Preferably, the vector has the following manner of arrangement and connection from upstream to downstream: nifJ-cleav-nifV-cleav-nifW, nifJ-cleav-nifV-cleav-nifZ, or nifJ-cleav-nifV-cleav-nifW-cleav-nifZ, wherein cleav is a nucleotide sequence encoding a cleavage sequence recognized by a protease.

In some embodiments, the vector composition comprises a vector comprising coding sequences of WU and nifS genes, or comprises a vector comprising a coding sequence of WU gene and a vector comprising a coding sequence of nifS gene. In some embodiments, the vector composition comprises vectors comprising coding sequences of nifU and nifS genes, and preferably the vector has the following manner of arrangement and connection from upstream to downstream: nifU-cleav-nifS, wherein cleav is a nucleotide sequence encoding a cleavage sequence recognized by a protease.

In some embodiments of the vector compositions, the vector composition comprises the following vectors:

a) a vector with nifH-cleav-nifD-cleav-nifK;

b) a vector with nifE-cleav-nifN-linker-nifB;

c) a vector with nifU-cleav-nifS;

d) a vector with nifJ-cleav-nifV-cleav-nifW, or nifJ-cleav-nifV-cleav-nifZ; and

e) a vector with nifF-cleav-nifM-cleav-nifY,

wherein cleav is a nucleotide sequence encoding a cleavage sequence recognized by a protease, and linker is a nucleotide sequence encoding a linker.

In other embodiments, the vector composition comprises the following vectors:

a) a vector with nifH-cleav-nifD-cleav-nifK;

b) a vector with nifE-cleav-nifN-linker-nifB;

c) a vector with nifU;

d) a vector with nifS;

e) a vector with nifJ-cleav-nifV-cleav-nifW; and

f) a vector with nifF-cleav-nifM-cleav-nifY,

wherein cleav is a nucleotide sequence encoding a cleavage sequence recognized by a protease, and linker is a nucleotide sequence encoding a linker.

In some embodiments, the vector composition comprises the following vectors:

a) a vector with nifH-cleav-nifD-cleav-nifK;

b) a vector with nifE-cleav-nifN-linker-nifB;

c) a vector with nifU-cleav-nifS-cleav-nifV;

d) a vector with nifJ; and

e) a vector with nifF-cleav-nifM-cleav-nifY,

wherein cleav is a nucleotide sequence encoding a cleavage sequence recognized by a protease, and linker is a nucleotide sequence encoding a linker.

In some embodiments, the vector composition comprises the following vectors:

a) a vector with nifH-cleav-nifD-cleav-nifK;

b) a vector with nifE-cleav-nifN-linker-nifB;

c) a vector with nifU-cleav-nifS;

d) a vector with nifJ-cleav-nifV; and

e) a vector with nifF-cleav-nifM-cleav-nifY,

wherein cleav is a nucleotide sequence encoding a cleavage sequence recognized by a protease, and linker is a nucleotide sequence encoding a linker.

In some embodiments, the vector composition comprises the following vectors:

a) a vector with nifH-cleav-nifD-cleav-nifK;

b) a vector with nifE-cleav-nifN-linker-nifB;

c) a vector with nifU-cleav-nifS;

d) a vector with nifJ-cleav-nifV-cleav-nifW-cleav-nifZ; and

e) a vector with nifF-cleav-nifM-cleav-nifY,

wherein cleav is a nucleotide sequence encoding a cleavage sequence recognized by a protease, and linker is a nucleotide sequence encoding a linker.

In some embodiments, the vector composition comprises the following vectors:

a) a vector with nifH-cleav-nifD-cleav-nifK;

b) a vector with nifE-cleav-nifN-linker-nifB;

c) a vector with nifU-cleav-nifS;

d) a vector with nifJ;

e) a vector with nifF; and

f) a vector with nifV-cleav-nifW-cleav-nifM-cleav-nifY,

wherein cleav is a nucleotide sequence encoding a cleavage sequence recognized by a protease, and linker is a nucleotide sequence encoding a linker.

In some embodiments, the vector composition comprises the following vectors:

a) a vector with nifH-cleav-nifD-cleav-nifK;

b) a vector with nifE-cleav-nifN-linker-nifB;

c) a vector with nifU;

d) a vector with nifS;

e) a vector with nifJ;

f) a vector with nifF; and

g) a vector with nifV-cleav-nifW-cleav-nifM-cleav-nifY,

wherein cleav is a nucleotide sequence encoding a cleavage sequence recognized by a protease, and linker is a nucleotide sequence encoding a linker.

Host Cell, Transformation Method and Use

In one aspect, the invention relates to a host cell comprising a vector or a vector composition of the invention.

In another aspect, the invention relates to a method of transforming a host cell comprising a step of transducing or transfecting the host cell with a vector or a vector composition of the invention.

Nucleic acids, such as vectors or expression vectors, can be delivered to prokaryotic and eukaryotic cells by various methods known in the art. Methods for delivering nucleic acids into cells include, but are not limited to, various chemical, electrochemical and biological methods such as heat shock transformation, electroporation, transfection such as liposome-mediated transfection, DEAE-Dextran-mediated transfection or calcium phosphate transfection. In addition, a method such as treating a recipient cell with calcium chloride to increase its permeability to DNA, and a method of preparing competent cells from cells at a growth stage and then transforming with DNA can be used. A method in which DNA recipient cells are made into protoplasts or spheroplasts (which can easily take up recombinant DNA), and then the recombinant DNA is introduced into the DNA recipient cells can also be used. The transformation method is not particularly limited, and those skilled in the art can select a suitable transformation method according to, for example, the host cell used and the type of vector or expression vector to be transformed.

In yet another aspect, the invention relates to use of a vector or a vector composition of the invention for transforming a host cell. By using a vector or a vector composition of the present invention to transduce a host cell, a complex biological system can be expressed in the host cell, such that the host cell has a function or trait corresponding to the complex biological system.

In any of the embodiments described above with respect to the host cell, the method of transforming a host cell and the use of transforming a host cell, the host cell can be a prokaryotic cell or a eukaryotic cell. In some embodiments, the prokaryotic cell can be selected from: Pseudomonas fluorescens, Bacillus subtilis, Pseudomonas protegens, Pseudomonas putida, Pseudomonas veronii, Pseudomonas taetrolens, Pseudomonas balearica, Pseudomonas stutzeri, Pseudomonas aeruginosa, Pseudomonas syringae, Bacillus amyloliquefaciens, Burkholderia phytofirmans, Gluconacetobacter diazotrophicus, Herbaspirillum seropedicae, Bacillus cereus. In some embodiments, the eukaryotic cell can be selected from, for example, a cell of the following species: Oryza sativa, Triticum aestivum, Zea mays, Sorghum bicolor, Setaria italica, Solanum tuberosum, Ipomoea batatas, Arachis hypogaea, Brassica napus, Malva farviflora, Sesamum indicum, Olea europaea, Elaeis guineensis, Saccharum officinarum, Beta vulgaris, Gossypium spp.

### EXAMPLES

The content of the invention will be further described below in combination with the examples. The examples of the present application take the nitrogen fixation system as an exemplary complex biological system, and describe exemplary embodiments for expressing a complex biological system in a host cell using the method, the vector and the vector composition of the present invention. It should be understood that the following examples are illustrative only and should not be considered as limiting the scope of the invention.

There has been a long-standing attempt in reducing dependence on industrial nitrogen fertilizers through engineering non-legume crops so that these crops can fix nitrogen from the atmosphere themselves. One solution is achieved by transferring nitrogen fixation (nif) genes to plant cells. The main challenge to achieve this goal is how to maintain the balanced expression of the many gene products involved in biological nitrogen fixation processes in host cells to achieve nitrogen fixation functions.

Nitrogenase is a complex enzyme consisting of two metalloprotein components: the Fe protein (dinitrogenase reductase) and the MoFe protein (dinitrogenase). Although only three genes nifH, nifD and nifK are required to encode the structural subunits of the enzyme, nitrogenase maturation requires the assembly and insertion of three different metal cofactors, in a complex multistep process. The functionality of the Fe protein is conferred by a [Fe₄S₄] cluster, (synthesized by NifU and NifS) that bridges the NifH subunits in the Fe protein homodimer and is also dependent on the maturase protein, NifM. The mature MoFe protein holoenzyme is a heterotetramer formed from the NifD (α) and NifK (β) structural subunits that contains an [Fe₈S₇] cluster at the α-β interfaces, known as the P cluster, and a complex heterometalic co-factor, known as Fe Mo-co that has an interstitial carbon atom at its core and also contains an organic moiety, homocitrate [Fe₇—S₉—C—Mo-homocitrate]. The assembly pathway for FeMo-co biosynthesis, which contains one of the most complex heterometal clusters in biology, is highly complex, requiring at least 9 nif genes in vivo. The heterotetramer formed by NifEN, which is structurally and functionally related to NifDK, plays a crucial role in the FeMo-co maturation pathway. Maintaining the stoichiometry of the NifEN and NifDK tetrameric complexes and the requirement to balance expression ratios of all the nif gene products required for nitrogenase synthesis and activity is a vital prerequisite for engineering expression of nitrogenase in non-diazotrophic hosts.

In this study, we select a representative nitrogen fixation system from Klebsiella oxytoca to design a polyprotein-based expression strategy for stoichiometric expression of components required for the biosynthesis and activity of nitrogenase.

### Example 1. Evaluation of Nif Components for Polyprotein Assembly

To utilize the polyprotein-based strategy, we first evaluated the expression levels of the individual components of the nitrogen fixation system to determine which proteins are suitable for grouping into one group for stoichiometric expression. Secondly, when expressed as a fusion protein and after been cleaved by a protease, there will be a residual sequence at the N-terminals or C-terminal or both ends (which depends on the relative position of the coding sequence of the protein in the fusion expression vector) of the protein. The tolerance of each gene product to the presence of a residual sequence at the N-terminal or C-terminal was evaluated to arrange the individual coding sequences in the fusion expression vector. In this example, a tobacco etch virus protease is used as an exemplary protease.

In this example, the expression level of each nif gene in its native operon location is quantified as follows: each nif gene was fused in-frame to the lacZYA reporter and the resultant plasmids were co-transformed with plasmid pKU7017 into E. coli strain JM109 to measure β-galactosidase activity under diazotrophic conditions. The expression level of the nifH gene is set to 100%, and the relative expression level of each nitrogen-fixing gene is shown in Table 1.

TABLE 1
Relative expression level of nif genes

| nif gene | Relative expression level |
|---|---|
| H | 100 ± 11 |
| D | 55 ± 10 |
| K | 45 ± 8 |
| T | 8 ± 0 |
| Y | 8 ± 4 |
| E | 23 ± 2 |
| N | 27 ± 5 |
| X | 19 ± 2 |
| B | 16 ± 4 |
| Q | 1 ± 0 |
| U | 8 ± 2 |
| S | 16 ± 4 |
| V | 9 ± 2 |
| W | 6 ± 3 |
| Z | 6 ± 2 |
| M | 2 ± 1 |
| J | 31 ± 9 |
| F | 5 ± 0 |

Although the assay above does not take into account the stability of the native nif-encoded proteins, we observed that the ratio of nifH to nifDK expression was 2:1, which was consistent with the ratio for their respective accumulated protein products in Azotobacter. The relative expression levels of the above nif genes indicate that in the step of grouping genes, the coding sequences of nifH, nifD, and nifK (expression levels 100:55:45); nifE, nifN, and nifB (expression levels 23:27:16); nifU and nifS (expression levels 8:16); and nifF, nifM and nifY (expression levels 5:2:8) are each grouped into one group, and the remaining nifJ, nifV and optionally nifW and nifZ (expression levels 31:9:6:6) were grouped into one group, and a fusion expression vector was designed and constructed for each group of genes.

As described above, since cleavage residual sequences are generated at two ends of the cleavage products after TEVp cleavage, and the effect of such residual sequences on the activity of the proteins may introduce additional constraints on constructions of the fusion expression vectors, we further tested whether the activity of each protein product of nif genes would be affected in the presence of a cleavage residual sequence at its C-terminal, that is, the tolerance of the protein component to the residual sequence. Specifically, each nif gene carrying the coding sequence of the extended ENLYFQ-tail was used to replace the native gene in the operon-based biobrick system, and the acetylene reduction activity of each replaced gene was measured. The results are shown in Table 2, wherein the tolerance is shown as the activity of a gene carrying the coding sequence of the additional ENLYFQ-tail against the activity exhibited by the native genes in the biobrick system in E. coli JM109 (100%).

TABLE 2
Tolerance results for protease cleavage residual sequences

| nif gene | Residual sequence tolerance |
|---|---|
| H | 97 ± 6 |
| D | 89 ± 9 |
| K | 1 ± 0 |
| T | Not Determined |
| Y | 104 ± 7 |
| E | 85 ± 5 |
| N | 90 ± 9 |
| X | 106 ± 5 |
| B | 71 ± 8 |
| Q | 80 ± 3 |
| U | 85 ± 2 |
| S | 97 ± 6 |
| V | 117 ± 7 |
| W | 103 ± 6 |
| Z | 116 ± 9 |
| M | 126 ± 7 |
| J | 87 ± 5 |
| F | 90 ± 11 |

The results above revealed that NifK cannot tolerate the cleavage residual sequence at its C-terminal and therefore can only be located at the C-terminal of a polyprotein, that is, when constructing a fusion expression vector, the coding sequence of nifK was required to be located downstream of the coding sequences of other genes. The other components were tolerant to the residual sequence at C-terminal, although the activity of NifB was reduced by about 30%.

In addition, to minimalize the biological nitrogen fixation system and simplify the reassembly and arrangement of genes encoding polyproteins, the nifT, nifX, nifW, and nifZ genes may be omitted, which are not essential for biological nitrogen fixation in E. coli, as mutations in these genes do not influence nitrogen fixation activity. Further, nifQ may also be omitted because the function of its gene product can be recovered in the presence of a high concentration of molybdenum.

According to the results of the relative expression levels of genes and the results of the tolerance of each component to the protease cleavage sequence, the genes were grouped and a fusion expression vectors were constructed, in which the coding sequence of each gene was separated by a nucleotide sequence encoding the cleavage site recognized by TEVp. The constructs were annotated as nifH\check{o}D\check{o}K, nifE\check{o}N\check{o}B, nifU\check{o}S, nifF\check{o}M\check{o}Y, and nifJ\check{o}V, nifJ\check{o}V\check{o}W, nifJ\check{o}V\check{o}Z or nifJ\check{o}V\check{o}W\check{o}Z, where \check{o} indicates the nucleotide sequence encoding the TEVp recognition and cleavage sequence.

### Example 2. Activity Based Test

The functionality of polyproteins expressed by the fusion expression vectors constructed according to the grouping above, both before and after TEVp cleavage, was determined by measuring nitrogen fixation activity exhibited by each fusion expression vector when complemented with other native nif genes. Protease cleavage was achieved by introducing a cassette for expressing TEVp under the control of the P_{tac} promoter after induction with IPTG. TEVp expression did not influence the functionality of native nif gene products.

Acetylene reduction assay is generally used to determine the activity of nitrogenase as nitrogenase has the property of being able to catalyze the reduction of acetylene to ethylene. The measurement method used in this example is as follows: the construct to be tested was introduced into E. coli JM109 strain and cultured at 30° C. for 16 hours; single colonies were picked and inoculated into KPM-HN liquid culture medium, and cultured at 30° C. overnight; an appropriate amount of bacteria solution was collected and resuspended into 2 mL KPM-LN liquid culture medium to a final OD₆₀₀ of ˜0.3; the culture solution was transferred to a 20 mL anaerobic tube and air was repeatedly evacuated-inflated three times after the anaerobic tube was sealed to exhaust the air in the tube, and then filled with inert gas argon (Ar); after anaerobic culture at 30° C. for 6-8 h, 2 mL acetylene gas was added and the area of the generated ethylene peak, which represented nitrogenase activity, was detected ˜16 h later with a SHIMADZU GC-2014 gas chromatograph. The final nitrogenase activity measurement data is the average of three or more repeated experiments.

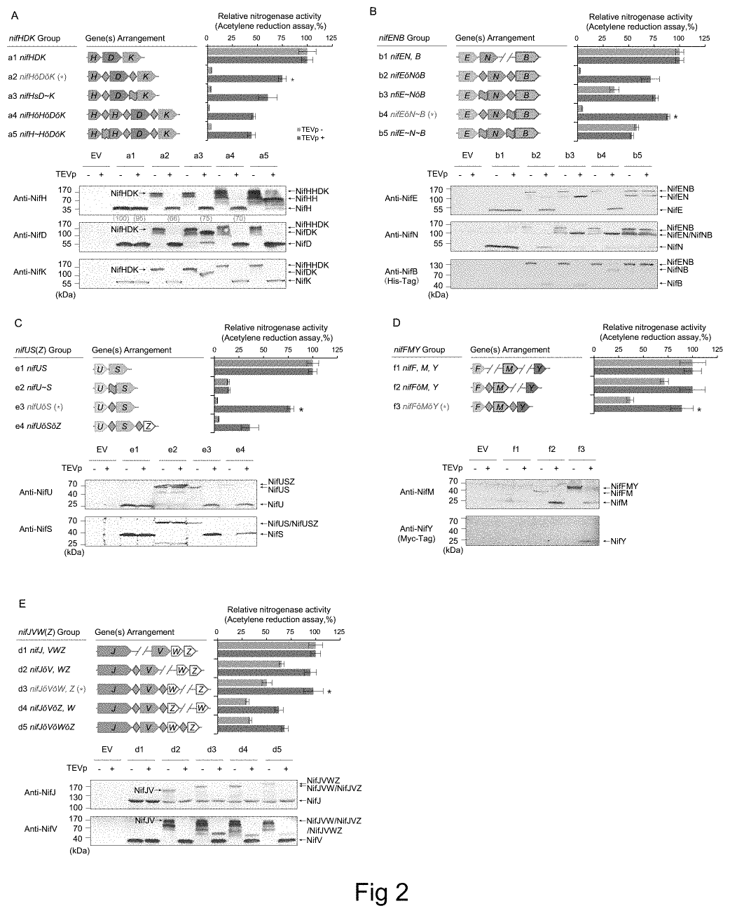

When assayed for acetylene reduction, the products of nifil\check{o}D\check{o}K gene, expressed from the nifH promoter, showed 75% nitrogenase activity after cleavage of its polyprotein product when expression of TEVp was induced, with a similar NifD: NifK protein stoichiometry to that expressed from the native genes. As NifB showed relative weak tolerance (71%, see table 2) to the C-terminal ENLYFQ-tail, we arranged the coding sequence of the nifB gene downstream of the construct, and the resultant nifE\check{o}N\check{o}B gene had 72% of nitrogenase activity when TEVp was expressed (FIG. 2B). For the nifU\check{o}S construct, native levels of NifU and NifS were restored after cleavage using this fusion expression vector and nitrogenase activity was recovered to 82% (FIG. 2C). In addition, the components generated after cleavage of the polyproteins encoded by nifF\check{o}M\check{o}Y by protease exhibited 89% of the nitrogenase activity (FIG. 2D). For the nifJ\check{o}V construct, we observed that the polyprotein product expressed by the fusion expression vector was active even prior to protease cleavage, as a fusion protein, resulting in 65% nitrogenase activity, which increased to 95% after cleavage with TEVp. The above results indicate that NifVJ can function of the two components as a fusion protein. In the fusion expression vector, the coding sequences can be directly linked in-frame or linked via a nucleotide sequence encoding a linker.

In order to further optimize activity, we carried out additional regrouping of genes encoding polyproteins and also tested the incorporation of fused genes as a means to simplify the ensembles. To further optimize NifHDK construct, we incorporated fusion proteins with different linkers. Fused NifD˜K protein (wherein “˜” represents a linker) showed broad tolerance to different lengths of GGGGS linkers, with a maximum activity of 91% when a 5×GGGGS linker was added.

For the nifENB group, functional fusions of NifE˜N and NifN˜B were obtained using 5×GGGGS and 3×GGGGS linkers, which exhibited an activity of 91% and 115% respectively. We thus replaced the corresponding part in the nifE\check{o}N\check{o}B gene to generate three further assemblies (nifE˜N\check{o}B, nifE\check{o}N˜B, and nifE˜N˜B). The incorporation of either the NifE˜N or NifN˜B fusion genes resulted in higher nitrogenase activities (76% and 89% respectively) compared with the nifE\check{o}N\check{o}B gene. However, when all three genes were fused to express the NifE˜N˜B fusion protein, only 50% nitrogenase activity was obtained. This decrease may reflect the presence of truncated NifE˜N translation product when expressing fusion proteins from nifE˜N˜B.

We also attempted expression of NifU and NifS as a fusion protein and obtained 50% nitrogenase activity when linked with a 5×GGGGS linker. This fusion protein had a lower activity than that obtained after cleavage of the NifU\check{o}S polyprotein.

### Example 3. Assembly and Characterization of Complete Polyprotein-Based Nitrogenase Systems

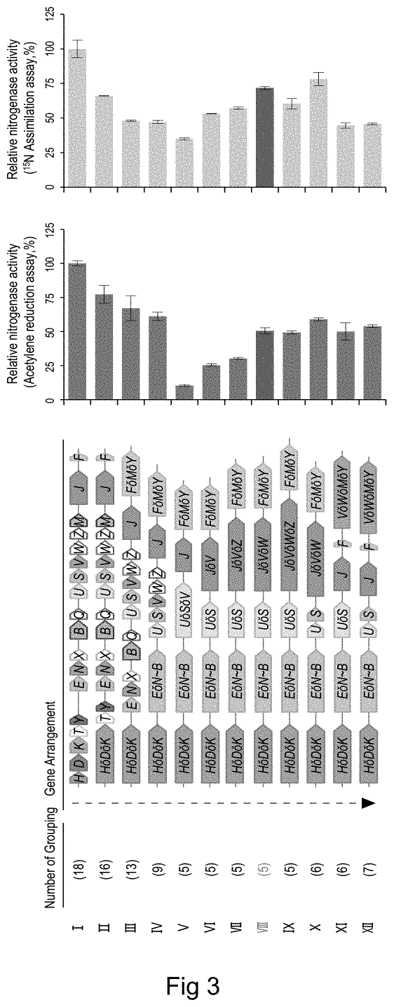

To assemble polyproteins into a functional Nif system, we sequentially replaced the native genes with fusion expression vectors described above. Combination of nifH\check{o}D\check{o}K with nifF\check{o}M\check{o}Y and nifE\check{o}N˜B (reducing the number of genes from 16 to 9) resulted in relatively small decreases in nitrogenase activity as measured by both acetylene reduction and ¹⁵N assimilation assays. However, replacement of the native nifUSVWZ genes with nifU\check{o}S\check{o}V (thus reducing the number of gene groups to five) resulted in a dramatic decrease in activity (10% of the native system) when acetylene reduction was measured (FIG. 3, group V). Nitrogenase activity was increased when nifV was removed from nifU\check{o}S\check{o}V and assigned to nifJ\check{o}V in the five gene group system as anticipated from the analysis of single polyproteins (FIG. 3, group VI).

In addition, although the nifW and nifZ gene products do not have a significant impact on the activity of our reconstituted system, previous studies suggest they are required for full activity of the MoFe protein. The decreased activity observed in the absence of nifW and nifZ prompted us to reconsider reintroduction of nifW and/or nifZ in the system. Considering the native locations and expression levels of nifW and nifZ, we combinated them with nifJ and nifV, designed additional constructs nifJ\check{o}V\check{o}Z, nifJ\check{o}V\check{o}W, and nifJ\check{o}V\check{o}W\check{o}Z to express their gene products as polyproteins. When the native genes were replaced with the above fusion expression vectors, the highest activity was obtained with the polyprotein expressing NifJVW (98%) (FIG. 3, group VIII), and no benefit was obtained by incorporating NifZ (FIG. 3, group VII and group IX). Similar activities were observed when these fusion expression vectors were complemented with the other four constructs encoding polyproteins, with nifJ\check{o}V\check{o}W again giving the highest level of activity (51% for acetylene reduction).

Quantitative analysis of protein levels by SRM mass spectrometry revealed that overall, the stoichiometry of most components from the polyprotein-based system matched remarkably well with the respective levels from the reconstituted operon-based system. This was particular for the NifHDK and NifENB proteins, where stoichiometry of these components is important for nitrogenase biosynthesis and activity (the level of NifK was determined by quantification of western blots).

Since the combined five-group (FIG. 3, group VIII) and six-group (FIG. 3, group X) polyprotein systems exhibited 72% and 75% ¹⁵N assimilation activity respectively, we anticipated that these expression systems could support diazotrophic growth by E. coli. The results show that in comparison with the initial single gene system, the arrangements of five-group and six-group (groups VIII and group X respectively) enabled E. coli to grow on solid media with N₂ as the sole nitrogen source, whereas groups IX and XI, which exhibited relative lower nitrogenase activates, grew less well under these conditions, although E. coli can also grow with N₂ as the sole nitrogen source (data not shown).

### Example 4. Application of the Polyprotein-Based Strategy to Eukaryotic Systems

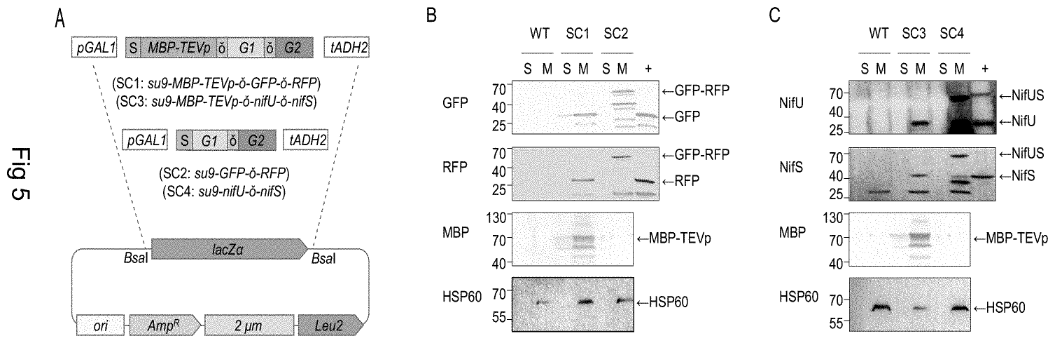

Eukaryotic organelles are considered to provide suitable locations for engineering nitrogen fixation system, as reported in the art that the expression of fully active nitrogenase components in yeast mitochondria and the detection of nitrogenase Fe protein activity in plant chloroplasts. As proof of principal for the expression, transport and cleavage of polyproteins in yeast mitochondria, we designed a construct encoding MBP-TEVp and two readily detectable fluorescent proteins, eGFP from Aequoria victoria and TurboRFP from Entacmaea quadricolor, encoding a single polyprotein (MBP-TEVp\check{o}GFP\check{o}RFP). Since proteins are translocated across mitochondrial membranes in an unfolded state, the TEVp would not be competent to initiate cleavage until protein folding occurs in the mitochondrial matrix. As a control for the self-cleavage reaction, a construct that lacked MBP-TEVp (GFP\check{o}RFP) was used. For mitochondrial targeting, the su9 leader sequence was added to the 5′ end of each fused gene, which were cloned downstream of the galactose inducible GAL1 promoter. After transformation of Saccharomyces cerevisiae, polyproteins carrying TEVp was expressed, efficiently translocated into mitochondria and cleaved by the protease into single components in mitochondria, as detected by western blotting (FIG. 5B). In contrast in the absence of TEVp, polyproteins were not digested.

Subsequently, two Nif proteins NifU and NifS, which can be stably expressed in yeast mitochondria without the existence of additional Nif proteins, were selected. Similar methods were used to construct fusion expression vectors and translocate polyproteins MBP-TEVp\check{o}NifU\check{o}S or NifU\check{o}S (FIG. 5A) into yeast mitochondria. Again, the fusion expreesion vector expressing MBP-TEVp enabled autonomous cleavage of the polyprotein to release the individual NifU and NifS components (FIG. 5C). Taken together, these results provide strong evidence that the polyprotein-based strategy provides an efficient solution for stoichiometric expression of individual components of the complex biological system in eukaryotic cells.

### Example 5. Application of the Polyprotein-Based Strategy to FeFe Nitrogen Fixation Systems

In previous studies, the inventors have constructed a simplified FeFe nitrogenase system (see WO 2015/192383 A1). The FeFe nitrogen fixation system comprises only 10 genes, namely nifU, nifS, nifV, nifB, nifJ and anfH, anfD, anfG and anfK. We also applied the grouping and expression strategies of the present invention to the FeFe nitrogenase system and resulted in the following groupings:

a) nifU-cleav-nifS;

b) nifJ-cleav-nifV-cleav-nifB;

c) nifF;

d) anfH;

e) anfD-linker-anfG; and

g) anfK,

wherein cleav is a nucleotide sequence encoding a cleavage sequence recognized by a protease, and linker is a nucleotide sequence encoding a linker.

The fusion expression vector constructed by employing this grouping manner can effectively express the active FeFe nitrogenase system in host cells.

1. A method for expressing a complex biological system comprising multiple genes encoding multiple components in a host cell, the method comprising:
a) determining the expression level of each gene in its native operon location;
b) grouping said genes according to the expression level of each gene determined in a), wherein each group comprises genes with similar expression levels;
c) constructing a fusion expression vector for each group of genes according to the grouping in b), wherein the fusion expression vector comprises coding sequences of all genes of its corresponding group, and wherein the coding sequences are directly linked in-frame, linked via a nucleotide sequence encoding a linker, or separated by a nucleotide sequence encoding a cleavage sequence recognized by a protease, thus obtaining a set of fusion expression vectors;
d) introducing the set of fusion expression vectors into a host cell to express a polyprotein from each expression vector;
e) expressing the protease in the host cell to cleave the polyproteins, wherein components encoded by coding sequences directly linked or linked via a nucleotide sequence encoding a linker are expressed as a fusion protein, and wherein components encoded by coding sequences separated by a nucleotide sequence encoding the cleavage sequence are released after protease cleavage.

2. The method of claim 1, wherein step c) further comprises testing the activity of the components encoded by genes in each group when expressed as a fusion protein, wherein coding sequences of two or more components that are capable of maintaining the activity of each component when expressed as fusion proteins are directly linked in-frame, or linked via a nucleotide sequence encoding a linker, and other coding sequences are separated by a nucleotide sequence encoding a cleavage sequence recognized by a protease,
wherein being capable of maintaining the activity of each component when expressed as fusion proteins means that when expressed as a fusion protein, the activity of each component is at least 30%, at least 40%, at least 50%, at least 60%, at least 70%, at least 75%, at least 80%, or at least 90% of its activity when expressed as a single protein.

3-4. (canceled)

5. The method of claim 2, wherein the activity is an enzymatic activity.

6. The method of claim 1, wherein step c) further comprises a step of arranging coding sequences in a construct, the step comprising testing each component for its tolerance in the presence of a residual sequence at the N-terminal or C-terminal after protease cleavage, wherein for a component with low tolerance in the presence of a residual sequence at the N-terminal, its coding sequence is arranged upstream of the coding sequences of other components; for a component with low tolerance in the presence of a residual sequence at the C-terminal, its coding sequence is arranged downstream of the coding sequences of other components; when there are two or more components with low tolerance in the presence of a residual sequence at the N-terminal in one group, only one of them is retained and its coding sequence is arranged upstream of the coding sequences, and other components with low tolerance in the presence of a residual sequence at the N-terminal are grouped into other groups; when there are two or more components with low tolerance in the presence of a residual sequence at the C-terminal in one group, only one of them is retained and its coding sequence is arranged downstream of the coding sequences, and other components with low tolerance in the presence of a residual sequence at the C-terminal are grouped into other groups,
wherein a component with low tolerance in the presence of a residual sequence at the N-terminal or C-terminal is defined as that the activity of the component is reduced by at least 10%, at least 20%, at least 30%, at least 40%, at least 50%, at least 60%, at least 70%, at least 80%, or at least 90% in the presence of a residual sequence at its N-terminal or C-terminal,
or
wherein a component with low tolerance in the presence of a residual sequence at the N-terminal or C-terminal is defined as: (activity in the presence of a residual sequence/activity in the absence of a residual sequence %)ⁿ is less than 30%, less than 40%, less than 50%, less than 60%, less than 70%, less than 80%, or less than 90%, wherein n is the number of genes of said complex biological system.

7-9. (canceled)

10. The method of claim 1, wherein the method further comprises adjusting the copy number of coding sequences so that genes originally with different expression levels achieve similar expression levels and are grouped into one group.

11. The method of claim 1, wherein each of the fusion expression vectors has a native expression control sequence of one of genes in its corresponding group or an expression control sequence having a similar expression level therewith.

12. The method of claim 1, wherein the protease is selected from the group consisting of thrombin, Factor Xa, enterokinase, Tobacco Etch Virus (TEV) protease, PreScission and HRV 3C protease.

13. (canceled)

14. The method of claim 1, wherein the host cell is a prokaryotic cell or a eukaryotic cell.

15. The method of claim 14, wherein the prokaryotic cell is selected from the group consisting of Pseudomonas fluorescens, Bacillus subtilis, Pseudomonas protegens, Pseudomonas putida, Pseudomonas veronii, Pseudomonas taetrolens, Pseudomonas balearica, Pseudomonas stutzeri, Pseudomonas aeruginosa, Pseudomonas syringae, Bacillus amyloliquefaciens, Burkholderia phytofirmans, Gluconacetobacter diazotrophicus, Herbaspirillum seropedicae, Bacillus cereus.

16. The method of claim 14, wherein the eukaryotic cell is selected from the cell of the following species: Oryza sativa, Triticum aestivum, Zea mays, Sorghum bicolor, Setaria italica, Solanum tuberosum, Ipomoea batatas, Arachis hypogaea, Brassica napus, Malva farviflora, Sesamum indicum, Olea europaea, Elaeis guineensis, Saccharum officinarum, Beta vulgaris, Gossypium spp.

17. The method of claim 1, wherein the complex biological system is selected from: alkane degradation pathway, nitrogen fixation system, polychlorinated biphenyl degradation system, bioplastic biosynthetic system, nonribosomal peptide biosynthetic system, polyketide biosynthetic system, terpenoid biosynthetic system, oligosaccharide biosynthetic system, indolocarbazole biosynthetic system.

18. (canceled)

19. The method of claim 17, wherein the complex biological system is a nitrogen fixation system and wherein the nitrogen fixation system comprises the following genes: nifH, nifD, nifK, nifY, nifE, nifN, nifX, nifB, nifU, nifU, nifS, nifV, nifM, nifJ, nifF and optionally nifT; nifX, nifQ, nifW, nifZ.

20. The method of claim 19, wherein the nitrogen fixation system is from Klebsiella oxytoca.

21. The method of claim 1, wherein the genes are grouped into three to seven groups.

22. (canceled)

23. The method of claim 19, wherein the following genes are grouped into one group: nifH, nifD, nifK, and wherein the fusion expression vector comprising the coding sequences of nifH, nifD, nifK genes has the following manner of arrangement and connection from upstream to downstream: nifH-cleav-nifD-cleav-nifK, wherein cleav is a nucleotide sequence encoding a cleavage sequence recognized by a protease.

24. (canceled)

25. The method of claim 19, wherein the following genes are grouped into one group: nifE, nifN, nifB, and wherein the fusion expression vector comprising the coding sequences of nifE, nifN, nifB genes has the following manner of arrangement and connection from upstream to downstream: nifE-cleav-nifN-linker-nifB, wherein cleav is a nucleotide sequence encoding a cleavage sequence recognized by a protease, and linker is a nucleotide sequence encoding a linker.

26-27. (canceled)

28. The method of claim 19, wherein the following genes are grouped into one group: nifF, nifM, nifY, and wherein the fusion expression vector comprising the coding sequences of nifF, nifM, nifY genes has the following manner of arrangement and connection from upstream to downstream: nifF-cleav-nifM-cleav-nifY, wherein cleav is a nucleotide sequence encoding a cleavage sequence recognized by a protease.

29. (canceled)

30. The method of claim 19, wherein the following genes are grouped into one group: nifJ, nifV and optionally nifW, nifZ, and wherein the fusion expression vector comprising the coding sequences of nifJ, nifV and optionally nifW, nifZ genes has the following structures from upstream to downstream: nifJ-cleav-nifV-cleav-nifW, nifZ-cleav-nifV-cleav-nifZ, or nifJ-cleav-nifV-cleav-nifW-cleav-nifZ, wherein cleav is a nucleotide sequence encoding a cleavage sequence recognized by a protease.

31. (canceled)

32. The method of claim 19, wherein nifU and nifS genes are grouped into one group, or nifU and nifS are expressed as independent genes, and wherein nifU and nifS genes are grouped into one group and the fusion expression vector comprising the coding sequences of nifU and nifS genes has the following manner of arrangement and connection from upstream to downstream: nifU-cleav-nifS, wherein cleav is a nucleotide sequence encoding a cleavage sequence recognized by a protease.

33. (canceled)

34. The method of claim 19, wherein the coding sequences of nifH, nifD, nifK, nifY, nifE, nifN, nifB, nifU, nifS, nifV, nifM, nifJ, nifF and optionally nifW, nifZ are cloned into five fusion expression vectors in the following manner of arrangement and connection:
a) nifH-cleav-nifD-cleav-nifK;
b) nifE-cleav-nifN-linker-nifB;
c) nifU-cleav-nifS;
d) nifJ-cleav-nifV-cleav-nifW, or nifJ-cleav-nifV-cleav-nifZ; and
e) nifF-cleav-nifM-cleav-nifY,
wherein cleav is a nucleotide sequence encoding a cleavage sequence recognized by a protease, and linker is a nucleotide sequence encoding a linker.

35. The method of claim 19, wherein the coding sequences of nifH, nifD, nifK, nifY, nifE, nifN, nifB, nifU, nifS, nifV, nifM, nifJ, nifF and nifW are cloned into six fusion expression vectors in the following manner of arrangement and connection:
a) nifH-cleav-nifD-cleav-nifK;
b) nifE-cleav-nifN-linker-nifB;
c) nifU;
d) nifS;
e) nifJ-cleav-nifV-cleav-nifW; and
f) nifF-cleav-nifM-cleav-nifY,
wherein cleav is a nucleotide sequence encoding a cleavage sequence recognized by a protease, and linker is a nucleotide sequence encoding a linker.

36-116. (canceled)
